(19) United States
(12) Patent Application Publication
(10) Pub. No.: US 2008/0085304 A1
(43) Pub. Date: Apr. 10, 2008

(54) Robust sustained release formulations

(75) Inventors: Anand R. Baichwal, Wappingers Falls, NY (US); Kevin Fitzmaurice, Middlebury, CT (US); Steve Labudzinski, Poughkeepsie, NY (US)

Correspondence Address: WILMERHALE/BOSTON, 60 STATE STREET, BOSTON, MA, 02109, US

(73) Assignee: Penwest Pharmaceuticals Co., Wappingers, NY (US)

(21) Appl. No.: 11/545,954

(22) Filed: Oct. 10, 2006

(51) Int. Cl. A61K 9/52; A61K 9/14; A61K 9/26

(52) U.S. Cl. 424/457; 424/469; 424/485; 424/500

(57) ABSTRACT

Robust sustained release formulations, solid dosage forms comprising robust sustained release formulations, and methods for making and using these formulations and solid dosage forms are provided. Robustness of the sustained release formulation is related to the particle size of the hydrophilic gum. Sustained release formulations resist dose-dumping when ingested with alcohol. The formulations are useful for treating a patient suffering from a condition, e.g., pain. The formulations comprise at least one drug. In one embodiment, the drug is an opioid, e.g., oxymorphone.

### 1. FIELD OF THE INVENTION

The invention provides robust sustained release pharmaceutical formulations and methods for making and using same. The formulations of the invention comprise at least one drug and a sustained release delivery system.

### 2. BACKGROUND OF THE INVENTION

Sustained release drug formulations often contain higher amounts of drugs than immediate release formulations. Functionality and safety of a sustained release formulation are based on a known controlled rate of drug release from the formulation over an extended period of time after administration, such as 8-24 hours. The drug release profile of a formulation often depends on the chemical environment of the sustained release formulation, for example, on pH, ionic strength and presence of solvents such as ethanol.

The relatively high amount of drug that is present in a sustained release formulation can, in some instances, harm a patient if the formulation releases the drug at a rate that is faster than the intended controlled release rate. If the formulation releases the drug at a rate that is slower than the intended controlled release rate, the therapeutic efficacy of the drug can be reduced.

In most cases, failure of a sustained release formulation results in a rapid release of the drug into the bloodstream. This rapid release is generally faster than the intended sustained release of the drug from the formulation, and is sometimes referred to as “dose dumping.”

Dose dumping can create severe consequences for a patient, including permanent harm and even death. Examples of drugs that can be fatal if the therapeutically beneficial dose is exceeded, e.g., by dose dumping, include pain medications such as opioids.

Oral dosage formulations are often taken with a commonly available beverage, such as water, juice, a carbonated beverage or occasionally an ethanol-containing beverage. An ethanol-containing beverage is commonly referred to as an alcoholic beverage, liquor, or simply alcohol. As used herein, “alcohol” refers to ethanol, or an ethanol-containing (“alcoholic”) beverage such as beer, wine, and hard liquors such as vodka, rum, or whiskey. Dose dumping in the presence of ethanol creates a safety concern because of the likelihood that a patient will ingest the formulation with an alcoholic beverage. This can be exacerbated where the drug may interact with the alcohol. An additional safety concern is that a patient will consume alcoholic beverages while being treated with the drug in the formulation, even if the patient does not ingest the formulation at the same time as an alcoholic beverage.

Patients who desire to abuse a drug, for example a drug that causes a euphoric effect, may want to intentionally induce dose dumping in order to magnify the euphoric effect of the drug. Furthermore, a person wanting to abuse a drug might already be abusing alcohol, which increases the likelihood of the sustained release formulation of the drug to be ingested or taken concurrently with an alcoholic beverage.

In 2005, several drugs were either withdrawn from the market or had their warning labels enhanced because of the effects of ethanol on the sustained release formulations of the drug.

For instance, the United States Food and Drug Administration (FDA) asked Purdue Pharma to withdraw Palladone® (hydromorphone hydrochloride) extended release capsules from the market because a study showed that when Palladone is taken with alcohol, its extended release formulation is damaged and can dose dump (c.f. FDA Press Release of Jul. 13, 2005). FDA further warned that taking Palladone® with a single alcoholic drink could have fatal consequences for the patient.

Alpharma issued a press release reporting that FDA had requested it to expand warning information regarding alcohol in the labeling for KADIAN® (c.f. Alpharma press release of Jul. 22, 2005). The enhanced warning was a result of in vitro studies showing that the extended release characteristics of KADIAN® are compromised in the presence of alcohol.

AVINZA® (morphine sulfate extended-release capsules) was found to have an increased risk of dose dumping when taken with ethanol. In vitro studies performed by the FDA showed that when AVINZA 30 mg was mixed with 900 mL of buffer solutions containing ethanol (20% and 40%), the dose of morphine that was released was alcohol concentration-dependent, leading to a more rapid release of morphine, which in vivo could result in the absorption of a potentially fatal dose of morphine. As a result, during or around October, 2005, Ligand Pharmaceuticals Inc. revised several sections of the AVINZA® prescribing information to highlight and strengthen the warning that patients should not consume alcohol while taking AVINZA®. Additionally, patients were warned not to use prescription or non-prescription medications containing alcohol while on AVINZA® therapy.

FDA has also indicated that for future sustained release products, in vitro testing for alcohol-induced undermining of sustained release characteristics may be advisable as a routine characterization test. Furthermore, FDA's position is that for certain drugs (e.g., drugs with a narrow therapeutic index or dire consequences of high Cₘₐₓ or low Cₘᵢₙ), alcohol sensitive sustained release formulations should not be approved. FDA prefers that formulations be made ethanol-resistant by design, rather than simply a confirmation that dose dumping does not occur through an in vivo study. (c.f. Summary of FDA's position on alcohol-induced dose dumping as presented at the Pharmaceutical Sciences Advisory Committee Meeting Oct. 26, 2005)

According to the FDA, an in vivo alcohol resistance test is not the preferred approach due to potential harm the test could pose to a human subject. The preferred approach, according to the FDA, is an in vitro dissolution test in the presence of 40% ethanol. This approach may be preferred because the strength of most common “hard” liquors is about 80 proof, or about 40% ethanol. FDA is proposing classifying formulations into three groups: rugged, vulnerable and uncertain. At the Pharmaceutical Sciences Advisory Committee Meeting of Oct. 26, 2005, OPS (Office of Pharmaceutical Science) at the CDER (Center for Drug Evaluation and Research) personnel presented data showing that in a vulnerable formulation, a higher concentration of ethanol (e.g., 40%) is likely to trigger faster drug release than a lower concentration of ethanol (e.g., 20% or 4%). In FDA's example of a rugged formulation, the drug release from a formulation dissolved in 40% ethanol is actually slightly slower (although similar) compared to a control formulation dissolved in a medium without ethanol. (Presentations at the Pharmaceutical Sciences Advisory Committee Meeting Oct. 26, 2005)

Changes to product labeling (i.e., added warnings of the danger of taking the drug with alcohol) have only a limited effect and are not likely to deter a patient who intends to abuse the drug.

Pain is the most frequently reported symptom and it is a common clinical problem that confronts the clinician. Many millions of people in the United States suffer from severe pain that is chronically undertreated or inappropriately managed. The clinical usefulness of the analgesic properties of opioids has been recognized for centuries, and morphine and its derivatives have been widely used for analgesia for decades in a variety of clinical pain states.

Oxymorphone HCl (14-hydroxydihydromorphinone hydrochloride) is a semi-synthetic phenanthrene-derivative opioid agonist, used in the treatment of acute and chronic pain, with analgesic efficacy comparable to other opioid analgesics. Oxymorphone is currently marketed as an injection (1 mg/ml in 1 ml ampules) for intramuscular, subcutaneous, and intravenous administration. At one time, a 10 mg oral immediate release tablet formation of oxymorphone HCl was marketed. Oxymorphone HCl is metabolized principally in the liver and undergoes conjugation with glucuronic acid and reduction to 6-alpha and 6-beta hydroxy epimers.

An important goal of analgesic therapy is to achieve continuous relief of chronic pain. Regular administration of an analgesic is generally required to ensure that the next dose is given before the effects of the previous dose have worn off. Compliance with opioids increases as the required dosing frequency decreases. Non-compliance results in suboptimal pain control and poor quality of life outcomes. Scheduled rather than “as needed” administration of opioids is currently recommended in guidelines for their use in treating chronic non-malignant pain. Unfortunately, evidence from prior clinical trials and clinical experience suggests that the short duration of action of immediate release oxymorphone would necessitate administration every four hours in order to maintain optimal levels of analgesia in patients with chronic pain. Moreover, immediate release oxymorphone exhibits low oral bioavailability, because oxymorphone is extensively metabolized in the liver.

Because many drugs, e.g., opioids such as oxymorphone, can cause serious adverse effects or even death to a patient if the sustained release formulation fails, there is a need in the art for pharmaceutical formulations that are more robust or rugged, and therefore safer, when compared to currently available sustained release formulations.

Several sustained release formulations have been described in U.S. Pat. No. 5,399,358, the disclosure of which is incorporated by reference herein in its entirety. It has now been unexpectedly discovered that the particle size of hydrophilic gums, e.g., xanthan gum, affects the robustness and dissolution properties of sustained release formulations.

Citation of a reference in Section 2 of the application is not an admission that the reference is prior art.

### 3. SUMMARY OF THE INVENTION

The invention provides sustained release pharmaceutical formulations and solid dosage forms comprising the sustained release formulations. The invention also provides methods for treating a patient using the sustained release formulations and methods for preventing dose dumping, for example, by providing to patients a therapeutically effective amount of a sustained release drug formulation. The pharmaceutical formulations described herein are less likely to dose dump compared to conventional sustained release formulations, which makes them more rugged, safer, and applicable to a wide variety of drugs.

The invention further provides ethanol-resistant pharmaceutical formulations and methods for increasing drug safety and reducing the potential for drug abuse. This can be achieved by providing, prescribing and/or administering to patients an effective amount of an ethanol-resistant drug formulation. The ethanol-resistant drug formulations are safer and have less potential for abuse when compared to commercially available formulations because their sustained release dissolution profile in an aqueous solution or in an ethanol-containing solution is essentially the same. In one embodiment, the drug in the ethanol-resistant formulation comprises an opioid compound or a derivative thereof.

The invention also provides ethanol-resistant pharmaceutical formulations and methods for preventing dose dumping. This can be achieved by providing, prescribing and/or administering to patients an effective amount of an ethanol-resistant drug formulation. The ethanol-resistant pharmaceutical formulations described herein do not dose dump in the presence of beverage-strength ethanol. In one embodiment, the drug in the ethanol-resistant formulation comprises an opioid compound, a pharmaceutically acceptable salt of an opioid compound, or a derivative thereof.

In one aspect, the invention provides a sustained release formulation comprising: a drug; and a sustained release delivery system comprising a hydrophilic gum, a homopolysaccharide gum, and a pharmaceutical diluent, wherein at least about 30% of the hydrophilic gum used to make the sustained release formulation can pass through a #270 mesh sieve and the sustained release formulation releases less than about 70% of the drug within 2 hours after ingestion with either an ethanol-free or an ethanol-containing beverage.

In another aspect, the invention provides a sustained release formulation comprising: a drug; and a sustained release delivery system comprising a hydrophilic gum, a cationic cross-linking compound selected from monovalent cations, multivalent cations and salts, and a pharmaceutical diluent, wherein at least about 30% of the hydrophilic gum used to make the sustained release formulation can pass through a #270 mesh sieve and the sustained release formulation releases less than about 70% of the drug within 2 hours after ingestion with either an ethanol-free or an ethanol-containing beverage.

In some embodiments, the hydrophilic gum is a heteropolysaccharide gum. In some embodiments, the hydrophilic gum is xanthan gum.

In one embodiment, the sustained release delivery system further comprises a cationic cross-linking compound selected from monovalent cations, multivalent cations, and salts. In one embodiment, the cationic cross-linking agent is a sodium salt.

In yet another aspect, the invention provides a sustained release formulation comprising: a drug; and a sustained release delivery system comprising a hydrophilic gum, a homopolysaccharide gum, and a pharmaceutical diluent, wherein at least about 30% of the hydrophilic gum particles used to make the sustained release formulation are smaller than about 53 microns in diameter and the sustained release formulation releases less than 70% of the drug within 2 hours after ingestion with either an ethanol-free or an ethanol-containing beverage.

In still another aspect, the invention provides a sustained release formulation comprising: a drug; and a sustained release delivery system comprising a hydrophilic gum, a cationic cross-linking compound selected from monovalent cations, multivalent cations and salts, and a pharmaceutical diluent, wherein at least about 30% of the hydrophilic gum particles used to make the sustained release formulation are smaller than about 53 microns in diameter and the sustained release formulation releases less than 70% of the drug within 2 hours after ingestion with either an ethanol-free or an ethanol-containing beverage. In some embodiments, the sustained delivery system further comprises a hydrophobic polymer.

In some embodiments, the sustained release formulation further comprises an outer coating. In some embodiments, the outer coating comprises a hydrophobic polymer and/or a plasticizer.

In some embodiments, the drug is a water-soluble drug. In some embodiments, the drug is an anti-depressant, a drug used to treat bipolar disorder, panic disorder, epilepsy, migraine, and/or attention deficit hyperactivity disorder. In some embodiments, the drug is selected from the group consisting of alprazolam, lithium carbonate, divalproex sodium, neutral sulfate salts of dextroamphetamine and amphetamine with the dextro isomer of amphetamine saccharate and d,l-amphetamine aspartate monohydrate, tramadol hydrochloride, and other pharmaceutically acceptable salts of the active pharmaceutical ingredient thereof.

In some embodiments, the drug is an opioid, e.g., alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papaveretum, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, an ether thereof, an ester thereof, and a derivative thereof and a pharmaceutically acceptable salt thereof.

Additionally, the invention provides methods for making sustained release pharmaceutical formulations and solid dosage forms comprising the sustained release formulations.

In one aspect, the invention provides a method for making a sustained release formulation comprising: a drug; and a sustained release delivery system, wherein the sustained release delivery system comprises a hydrophilic gum, a homopolysaccharide gum, and a pharmaceutical diluent, the method comprising: providing the hydrophilic gum with at least a fraction of particles less than about 53 microns in diameter; granulating the hydrophilic gum, the homopolysaccharide gum and the pharmaceutical diluent to form granules; mixing the granules with the drug to form a granulated composition; and applying pressure to the granulated composition to make the formulation.

In another aspect, the invention provides a method for making a sustained release formulation comprising: a drug; and a sustained release delivery system, wherein the sustained release delivery system comprises a hydrophilic gum, a cationic cross-linking compound selected from monovalent cations, multivalent cations and salts, and a pharmaceutical diluent, the method comprising: providing the hydrophilic gum with at least a fraction of particles less than about 53 microns in diameter; granulating the hydrophilic gum, the homopolysaccharide gum and the pharmaceutical diluent to form granules; mixing the granules with the drug to form a granulated composition; and applying pressure to the granulated composition to make the formulation.

In some embodiments, providing comprises receiving, manufacturing, and/or processing the hydrophilic gum. In some embodiments, processing comprises measuring the size of at least a fraction of the hydrophilic gum particles and/or passing at least a fraction of the hydrophilic gum through a sieve. In some embodiments, the sieve is a #270 mesh sieve.

In some embodiments, the method for making the sustained release formulation and a solid dosage form further comprises applying an outer coating onto at least part of the sustained release formulation.

In some embodiments, granulating comprises mixing ingredients with a solution comprising water. In other embodiments, granulating comprises mixing ingredients with an alcohol solution, for example a solution comprising ethanol.

In one aspect, the invention provides a method for making a sustained release formulation comprising: a drug; and a sustained release delivery system, wherein the sustained release delivery system comprises a hydrophilic gum, a homopolysaccharide gum, and a pharmaceutical diluent, the method comprising: mixing the hydrophilic gum of average and/or mean particle size larger than about 53 microns in diameter, the homopolysaccharide gum and the pharmaceutical diluent with a solution comprising water to form granules; mixing the granules with drug to form a granulated composition; and applying pressure to the granulated composition to make the formulation.

In another aspect, the invention provides a method for making a sustained release formulation comprising: a drug; and a sustained release delivery system, wherein the sustained release delivery system comprises a hydrophilic gum, a cationic cross-linking compound selected from monovalent cations, multivalent cations and salts, and pharmaceutical diluent, the method comprising: mixing the hydrophilic gum of average and/or mean particle size larger than about 53 microns in diameter, the cationic cross-linking compound and the pharmaceutical diluent with a solution comprising water to form granules; mixing the granules with the drug to form a granulated composition; and applying pressure to the granulated composition to make the formulation.

In one embodiment, a method for making a sustained release formulation further comprises recording a dissolution profile of the sustained release formulation or a solid dosage form comprising the sustained release formulation in an ethanol-containing solution.

In one embodiment, the invention provides a method for relieving pain comprising administering to a patient a therapeutically effective amount of a sustained release formulation or a solid dosage form comprising a sustained release formulation described herein.

In another embodiment, the invention provides a method for treating a patient having a condition comprising administering to the patient a therapeutically effective amount of a sustained release formulation or a solid dosage form comprising a sustained release formulation described herein.

In another aspect, the invention provides a method for reducing dose dumping of a sustained release drug formulation comprising providing a patient a sustained release formulation described herein.

In yet another aspect, the invention provides a solid dosage form comprising a sustained release formulation described herein. In some embodiments, the solid dosage form is a powder, a granule, a tablet, or a capsule.

In one aspect, the sustained release formulation comprises from about 5 to about 80 mg of oxymorphone hydrochloride and about 80 mg to about 360 mg of a sustained release delivery system; wherein the sustained release delivery system comprises from about 8.3% to about 41.7% by weight locust bean gum from about 8.3% to about 41.7% by weight xanthan gum wherein at least about 30% of the xanthan gum particles can pass through a #270 mesh sieve; from about 20% to about 55% by weight dextrose, from about 5% to about 20% by weight calcium sulfate dihydrate, and from about 2% to about 10% ethyl cellulose, and the sustained release formulation releases less than 70% of the drug within 2 hours after ingestion with either an ethanol-free or an ethanol-containing beverage.

In another aspect, the sustained release formulation comprises from about 5 to about 80 mg of oxymorphone hydrochloride and from about 300 mg to about 420 mg of a sustained release delivery system; wherein the sustained release delivery system comprises from about 8.3% to about 41.7% by weight locust bean gum, from about 8.3% to about 41.7% by weight xanthan gum having at least about 30% of particles smaller than about 53 microns in diameter; from about 20% to about 55% by weight dextrose, from about 5% to about 20% by weight calcium sulfate dihydrate, and from about 2% to about 10% ethyl cellulose, and the sustained release formulation releases less than 70% of the drug within 2 hours after ingestion with either an ethanol-free or an ethanol-containing beverage.

In one embodiment, the sustained release formulation comprises about 20 mg of oxymorphone hydrochloride. In another embodiment, the sustained release formulation comprises about 160 mg of a sustained release delivery system. In yet another embodiment, the sustained release formulation comprises about 360 mg of a sustained release delivery system. In still another embodiment, the sustained release delivery system comprises about 25% locust bean gum, about 25% xanthan gum, about 35% dextrose, about 10% calcium sulfate dihydrate, and about 5% ethyl cellulose.

In another aspect, the invention provides a method of preventing dose-dumping of a drug in the presence of ethanol comprising: providing a patient who could consume ethanol while being treated with the drug an effective amount of the drug in the form of an ethanol-resistant sustained release formulation comprising: the drug; and a sustained release delivery system, the delivery system comprising at least one hydrophilic gum, at least one homopolysaccharide gum and at least one pharmaceutical diluent, wherein at least about 30% of the hydrophilic gum used to make the sustained release formulation can pass through a #270 mesh sieve and the sustained release formulation releases less than about 70% of the drug within 2 hours after ingestion with either an ethanol-free or an ethanol-containing beverage.

In one aspect, the invention provides a method of preventing dose-dumping of a drug in the presence of ethanol comprising: providing a patient who could consume ethanol while being treated with the drug an effective amount of the drug in the form of an ethanol-resistant sustained release formulation comprising: the drug; and a sustained release delivery system, the delivery system comprising at least one hydrophilic gum, at least one cationic cross-linking compound selected from monovalent metal cations, multivalent metal cations and salts, and at least one pharmaceutical diluent, wherein at least about 30% of the hydrophilic gum used to make the sustained release formulation can pass through a #270 mesh sieve and the sustained release formulation releases less than about 70% of the drug within 2 hours after ingestion with either an ethanol-free or an ethanol-containing beverage.

In another aspect, the invention provides a method of improving safety of a drug formulation comprising: providing a patient who could consume ethanol while being treated with the drug an effective amount of the drug in the form of an ethanol-resistant sustained release formulation comprising: the drug; and a sustained release delivery system, the sustained release delivery system comprising at least one hydrophilic gum, at least one homopolysaccharide gum and at least one pharmaceutical diluent, wherein the improvement in safety is a result of controlled hydrophilic gum particle size and ethanol-resistant sustained release properties of the formulation.

In yet another aspect, the invention provides a method of improving safety of a drug formulation comprising: providing a patient who could consume ethanol while being treated with the drug an effective amount of the drug in the form of an ethanol-resistant sustained release formulation comprising: the drug; and a sustained release delivery system, the delivery system comprising at least one hydrophilic gum, at least one cationic cross-linking compound selected from monovalent metal cations, multivalent metal cations and salts, and at least one pharmaceutical diluent, wherein the improvement in safety is a result of controlled hydrophilic gum particle size and ethanol-resistant sustained release properties of the formulation.

In one aspect, the invention provides a sustained release oxymorphone formulation comprising a sustained release delivery system and from about 5 mg to about 80 mg of oxymorphone, wherein after oral administration of a single dose to a patient with about 200 mL to about 300 mL of about 4% to about 40% ethanol the formulation provides a secondary peak of blood oxymorphone concentration about 12 hours after administration, and the formulation provides analgesia to the patient for at least about 12 hours after administration.

In some embodiments, the formulation comprises from about 20 mg to about 60 mg of oxymorphone or about 40 mg of oxymorphone. In one embodiment, the formulation is a solid dosage, for example, a tablet, a granule, a capsule or a powder.

In another aspect, the invention provides a sustained release oxymorphone formulation comprising a sustained release delivery system and from about 5 mg to about 80 mg of oxymorphone, wherein after oral administration of a single dose to a patient the formulation provides a maximum blood concentration of oxymorphone less than about 5 times higher when ingested with about 200 mL to about 300 mL of up to about 40% ethanol compared to when ingested without ethanol, and the formulation provides analgesia to the patient for at least about 12 hours after administration.

In one embodiment, the maximum blood concentration of oxymorphone is less than about 2.5 times higher when ingested with about 200 mL to about 300 mL of up to about 40% ethanol compared to when ingested without ethanol.

In some embodiments, the formulation comprises from about 20 mg to about 60 mg of oxymorphone or about 40 mg of oxymorphone. In one embodiment, the formulation is a solid dosage, for example, a tablet, a granule, a capsule or a powder.

In yet another aspect, the invention provides a sustained release oxymorphone formulation comprising a sustained release delivery system and from about 5 mg to about 80 mg of oxymorphone, wherein after oral administration of a single dose to a patient the formulation provides a ratio of the maximum blood concentration of oxymorphone when ingested with about 200 mL to about 300 mL of about 40% ethanol to the maximum blood concentration of oxymorphone when ingested after a high-fat meal without ethanol from about 0.5 to about 2, and the formulation provides analgesia to the patient for at least about 12 hours after administration.

In one embodiment, the ratio of the maximum blood concentration of oxymorphone when the formulation is ingested with about 200 mL to about 300 mL of about 40% ethanol to the maximum blood concentration of oxymorphone when the formulation is ingested after a high-fat meal without ethanol is from about 0.8 to about 1.5.

In some embodiments, the formulation comprises from about 20 mg to about 60 mg of oxymorphone or about 40 mg of oxymorphone. In one embodiment, the formulation is a solid dosage, for example, a tablet, a granule, a capsule or a powder.

In one aspect, the invention provides a sustained release oxymorphone formulation comprising a sustained release delivery system and from about 5 mg to about 80 mg of oxymorphone, wherein after oral administration of a single dose to a patient with about 200 mL to about 300 mL of about 4% to about 40% ethanol the formulation provides a maximum blood concentration of oxymorphone from about 0.1 ng/mL to about 15 ng/mL, and the formulation provides analgesia to the patient for at least about 12 hours after administration.

In some embodiments, the formulation provides a maximum blood concentration of oxymorphone from about 0.5 ng/mL to about 7.5 ng/mL or from about 1 ng/mL to about 4 ng/mL.

In one embodiment, the formulation comprises from about 10 mg to about 20 mg of oxymorphone and the formulation provides a maximum blood concentration of oxymorphone from about 0.3 ng/mL to about 3.2 ng/mL or from about 0.4 ng/mL to about 2.8 ng/mL.

In some embodiments, the formulation comprises about 10 mg of oxymorphone and the formulation provides a maximum blood concentration of oxymorphone from about 0.3 ng/mL to about 1.8 ng/mL or from about 0.5 ng/mL to about 1.5 ng/mL.

In another embodiment, the formulation comprises from about 20 mg to about 40 mg of oxymorphone and the formulation provides a maximum blood concentration of oxymorphone from about 0.5 ng/mL to about 7 ng/mL or from about 0.9 ng/mL to about 6 ng/mL.

In yet another embodiment, the formulation comprises about 20 mg of oxymorphone and the formulation provides a maximum blood concentration of oxymorphone from about 0.5 ng/mL to about 3.2 ng/mL or from about 0.75 ng/mL to about 2.8 ng/mL.

In one embodiment, the formulation comprises from about 40 mg to about 80 mg of oxymorphone and the formulation provides a maximum blood concentration of oxymorphone from about 1 ng/mL to about 15 ng/mL or from about 1.9 ng/mL to about 12 ng/mL.

In another embodiment, the formulation comprises about 40 mg of oxymorphone and the formulation provides a maximum blood concentration of oxymorphone from about 1 ng/mL to about 7 ng/mL or from about 1.4 ng/mL to about 5 ng/mL.

In yet another embodiment, the formulation comprises about 80 mg of oxymorphone and the formulation provides a maximum blood concentration of oxymorphone from about 3.5 ng/mL to about 15 ng/mL or from about 4 ng/mL to about 13 ng/mL.

In one aspect, the invention provides a sustained release oxymorphone formulation comprising a sustained release delivery system and from about 5 mg to about 80 mg of oxymorphone, wherein the formulation provides a minimum blood concentration of oxymorphone of at least about 0.013 ng/mL at about 12 hours after oral administration of a single dose to a patient with about 200 mL to about 300 mL of about 4% to about 40% ethanol, and the formulation provides analgesia to the patient for at least about 12 hours after administration.

In one embodiment, the formulation comprises about 5 mg of oxymorphone and provides a minimum blood concentration of oxymorphone of at least about 0.07 ng/mL.

In another embodiment, the formulation comprises about 10 mg of oxymorphone and provides a minimum blood concentration of oxymorphone of at least about 0.15 ng/mL.

In yet another embodiment, the formulation comprises about 20 mg of oxymorphone and provides a minimum blood concentration of oxymorphone of at least about 0.3 ng/mL.

In one embodiment, the formulation comprises about 40 mg of oxymorphone and provides a minimum blood concentration of oxymorphone of at least about 0.6 ng/mL.

In yet another embodiment, the formulation comprises about 80 mg of oxymorphone and provides a minimum blood concentration of oxymorphone of at least about 1.2 ng/mL.

In some embodiments, the formulation is a solid dosage form, for example, a tablet, a capsule, a granule, or a powder.

In one aspect, the invention provides a method of relieving pain comprising administering to a patient a sustained release oxymorphone formulation comprising a sustained release delivery system and from about 5 mg to about 80 mg of oxymorphone, wherein after oral administration of a single dose to the patient with about 200 mL to about 300 mL of about 4% to about 40% ethanol the formulation provides a secondary peak of blood oxymorphone concentration about 12 hours after administration, and the formulation provides analgesia to the patient for at least about 12 hours after administration.

In some embodiments, the formulation comprises from about 20 mg to about 60 mg of oxymorphone or about 40 mg of oxymorphone. In one embodiment, the formulation is a solid dosage, for example, a tablet, a granule, a capsule or a powder.

In another aspect, the invention provides a method of relieving pain comprising administering to a patient a sustained release oxymorphone formulation comprising a sustained release delivery system and from about 5 mg to about 80 mg of oxymorphone, wherein after oral administration of a single dose to a patient the formulation provides a maximum blood concentration of oxymorphone less than about 5 times higher when ingested with about 200 mL to about 300 mL of up to about 40% ethanol compared to when ingested without ethanol, and the formulation provides analgesia to the patient for at least about 12 hours after administration.

In one embodiment, the maximum blood concentration of oxymorphone is less than about 2.5 times higher when ingested with about 200 mL to about 300 mL of up to about 40% ethanol compared to when ingested without ethanol.

In some embodiments, the formulation comprises from about 20 mg to about 60 mg of oxymorphone or about 40 mg of oxymorphone. In one embodiment, the formulation is a solid dosage, for example a tablet, a granule, a capsule or a powder.

In yet another aspect, the invention provides a method of relieving pain comprising administering to a patient a sustained release oxymorphone formulation comprising a sustained release delivery system and from about 5 mg to about 80 mg of oxymorphone, wherein after oral administration of a single dose to a patient the formulation provides a ratio of the maximum blood concentration of oxymorphone when ingested with about 200 mL to about 300 mL of about 40% ethanol to the maximum blood concentration of oxymorphone when ingested after a high-fat meal without ethanol of about 0.5 to about 2, and the formulation provides analgesia to the patient for at least about 12 hours after administration.

In one embodiment, the ratio of the maximum blood concentration of oxymorphone when the formulation is ingested with about 200 mL to about 300 mL of about 40% ethanol to the maximum blood concentration of oxymorphone when the formulation is ingested after a high-fat meal without ethanol is from about 0.8 to about 1.5.

In some embodiments, the formulation comprises from about 20 mg to about 60 mg of oxymorphone or about 40 mg of oxymorphone. In one embodiment, the formulation is a solid dosage, for example, a tablet, a granule, a capsule or a powder.

In one aspect, the invention provides a method of relieving pain comprising administering to a patient a sustained release oxymorphone formulation comprising a sustained release delivery system and from about 5 mg to about 80 mg of oxymorphone, wherein after oral administration of a single dose to a patient with about 200 mL to about 300 mL of about 4% to about 40% ethanol the formulation provides a maximum blood concentration of oxymorphone from about 0.1 ng/mL to about 15 ng/mL, and the formulation provides analgesia to the patient for at least about 12 hours after administration.

In some embodiments, the formulation provides a maximum blood concentration of oxymorphone from about 0.5 ng/mL to about 7.5 ng/mL or from about 1 ng/mL to about 4 ng/mL.

In one embodiment, the formulation comprises from about 10 mg to about 20 mg of oxymorphone and the formulation provides a maximum blood concentration of oxymorphone from about 0.3 ng/mL to about 3.2 ng/mL or from about 0.4 ng/mL to about 2.8 ng/mL.

In some embodiments, the formulation comprises about 10 mg of oxymorphone and the formulation provides a maximum blood concentration of oxymorphone from about 0.3 ng/mL to about 1.8 ng/mL or from about 0.5 ng/mL to about 1.5 ng/mL.

In another embodiment, the formulation comprises from about 20 mg to about 40 mg of oxymorphone and the formulation provides a maximum blood concentration of oxymorphone from about 0.5 ng/mL to about 7 ng/mL or from about 0.9 ng/mL to about 6 ng/mL.

In yet another embodiment, the formulation comprises about 20 mg of oxymorphone and the formulation provides a maximum blood concentration of oxymorphone from about 0.5 ng/mL to about 3.2 ng/mL or from about 0.75 ng/mL to about 2.8 ng/mL.

In one embodiment, the formulation comprises from about 40 mg to about 80 mg of oxymorphone and the formulation provides a maximum blood concentration of oxymorphone from about 1 ng/mL to about 15 ng/mL or from about 1.9 ng/mL to about 12 ng/mL.

In another embodiment, the formulation comprises about 40 mg of oxymorphone and the formulation provides a maximum blood concentration of oxymorphone from about 1 ng/mL to about 7 ng/mL or from about 1.4 ng/mL to about 5 ng/mL.

In yet another embodiment, the formulation comprises about 80 mg of oxymorphone and the formulation provides a maximum blood concentration of oxymorphone from about 3.5 ng/mL to about 15 ng/mL or from about 4 ng/mL to about 13 ng/mL.

In another aspect, the invention provides a method of relieving pain comprising administering to a patient a sustained release oxymorphone formulation comprising a sustained release delivery system and from about 5 mg to about 80 mg of oxymorphone, wherein the formulation provides a minimum blood concentration of oxymorphone of at least about 0.013 ng/mL at about 12 hours after oral administration of a single dose to a patient with about 200 mL to about 300 mL of about 4% to about 40% ethanol, and the formulation provides analgesia to the patient for at least about 12 hours after administration.

In one embodiment, the formulation comprises about 5 mg of oxymorphone and provides a minimum blood concentration of oxymorphone of at least about 0.07 ng/mL.

In another embodiment, the formulation comprises about 10 mg of oxymorphone and provides a minimum blood concentration of oxymorphone of at least about 0.15 ng/mL.

In yet another embodiment, the formulation comprises about 20 mg of oxymorphone and provides a minimum blood concentration of oxymorphone of at least about 0.3 ng/mL.

In one embodiment, the formulation comprises about 40 mg of oxymorphone and provides a minimum blood concentration of oxymorphone of at least about 0.6 ng/mL.

In yet another embodiment, the formulation comprises about 80 mg of oxymorphone and provides a minimum blood concentration of oxymorphone of at least about 1.2 ng/mL. Sustained release formulations described herein can be used in therapy. Furthermore, sustained release formulations described herein can be used in the manufacture of a medicament for treatment of a condition. In one embodiment, the sustained release formulations described herein can be used for the manufacture of a medicament for relieving pain.

In some embodiments, the formulation is a solid dosage form, for example, a tablet, a capsule, a granule, or a powder.

These and other aspects and embodiments of the invention are described in detail herein.

### 4. DETAILED DESCRIPTION OF THE INVENTION

### 4.1. Definitions

As used herein, unless specifically indicated otherwise, the conjunction “or” is used in the inclusive sense of “and/or” and not the exclusive sense of “either/or.”

As used herein, the term “robust” refers to a property of a sustained release formulation that makes it less likely to have its dissolution profile substantially modified, injured, or otherwise fail. An example of a failure of a sustained release formulation is dose dumping. “Robust” and “rugged” are meant to be synonyms.

As used herein, the term “fine” refers to a particle size of a polymer having a diameter smaller than 53 microns, or alternatively, having particles capable of passing through a #270 mesh sieve.

As used herein, the term “dose dumping” refers to a rapid release of a drug or an active ingredient from a sustained release formulation into the bloodstream. This rapid release is generally faster than the sustained release of a drug from the formulation. Dose dumping also refers to a release having a peak concentration of the drug in the blood plasma higher than the peak concentration of the intended sustained release of the drug. Dose dumping can, in some instances, allow dangerous overdosing to occur, which can lead to fatal consequences.

As used herein, the term “sustained release” means that the drug is released from the formulation at a controlled rate so that therapeutically beneficial blood levels (but below toxic levels) of the drug are maintained over an extended period of time.

As used herein, terms “sustained release”, “extended release” and “controlled release” are meant to be synonyms, i.e., have identical meaning.

As used herein, the term “immediate release” means that the drug is released from the formulation in a short period of time, e.g., within about 4 hours after administration of the formulation.

As used herein, the term “AUC” refers to the area under the concentration-time curve.

As used herein, the term “Cₘₐₓ” refers to the maximum observed concentration.

As used herein, the term “RSD” refers to the relative standard deviation.

As used herein, the term “CI” refers to the confidence interval.

As used herein, the term “high-fat meal” refers to a meal wherein approximately 50 percent of total caloric content of the meal is derived from fat. An example of a high-fat meal is two eggs fried in butter, two strips of bacon, two slices of toast with butter, four ounces of hash brown potatoes and eight ounces of whole milk.

As used herein, the term “liquids” includes, for example, gastrointestinal fluids, aqueous solutions (such as those used for in vitro dissolution testing), and mucosas (e.g., of the mouth, nose, lungs, esophagus, and the like).

As used herein, the term “ethanol-resistant” refers to releasing less than 50% of an active ingredient (e.g., a drug) within one hour in a dissolution profile measurement by USP Procedure Drug Release USP 23 in 0.1N HCl and 40% ethanol solution.

As used herein, the term “drug” includes any pharmaceutically active chemical or biological compound, and any pharmaceutically acceptable salt thereof, used for alleviating symptoms, treating or preventing a condition.

Drugs suited for the robust sustained release formulations described herein include, but are not limited to, alprazolam (XANAX XR®), lithium carbonate (LITHOBID®), divalproex sodium (DEPAKOTE®), neutral sulfate salts of dextroamphetamine and amphetamine, with the dextro isomer of amphetamine saccharate and d,l-amphetamine aspartate monohydrate (ADDERALL XR®), tramadol hydrochloride (TRAMADOL ER®) and opioids such as morphine (AVINZA® and KADIAN®) and oxycodone (OXYCONTIN®).

As used herein, the term “opioid” includes stereoisomers thereof, metabolites thereof, salts thereof, ethers thereof, esters thereof and/or derivatives thereof (e.g., pharmaceutically acceptable salts thereof). The opioids may be mu-antagonists and/or mixed mu-agonists/antagonists. Exemplary opioids include alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papaveretum, pentazocine, phenadoxone, phenomorphan, phenazocine, phenoperidine, piminodine, piritramide, propheptazine, promedol, properidine, propiram, propoxyphene, sufentanil, tramadol, tilidine, stereoisomers thereof, metabolites thereof, salts thereof, ethers thereof, esters thereof, and/or derivatives thereof. In some embodiments, the opioid is morphine, codeine, hydromorphone, hydrocodone, oxycodone, dihydrocodeine, dihydromorphine, oxymorphone, 6-hydroxyoxymorphone (including 6-α-hydroxyoxymorphone and/or 6-β-hydroxyoxymorphone), or tramadol.

As used herein, the term “oxymorphone” includes oxymorphone, metabolites thereof, and derivatives thereof. Metabolites of oxymorphone include, for example, 6-hydroxyoxymorphone (e.g., 6-α-hydroxyoxymorphone and/or 6-β-hydroxyoxymorphone).

As used herein, the term “condition” includes any disease or a collection of symptoms that requires treatment with a drug. Exemplary conditions include panic disorder (with or without agoraphobia), bipolar disorder (manic depressive illness), acute manic or mixed episodes associated with bipolar disorder, epilepsy, migraine, attention deficit hyperactivity disorder (ADHD), depression and pain.

The pain can be minor to moderate, or moderate to severe. The pain can be acute or chronic. The pain can also be persistent and require continuous around-the-clock relief for an extended period of time. The pain can be associated with, for example, cancer, autoimmune diseases, infections, surgical traumas, or accidental traumas. The patient can be an animal, a mammal, or a human.

The drug may be in the form of any pharmaceutically acceptable salt known in the art.

Exemplary pharmaceutically acceptable salts include hydrochloric, sulfuric, nitric, phosphoric, hydrobromic, maleric, malic, ascorbic, citric, tartaric, pamoic, lauric, stearic, palmitic, oleic, myristic, lauryl sulfuric, napthalinesulfonic, linoleic, linolenic acid, and the like.

The robust sustained release formulations of drugs are administered in an amount sufficient to alleviate symptoms, treat or prevent a condition for an extended period of time, for example about 8 hours to about 24 hours, or for a period of about 12 hours to about 24 hours. The robust sustained release oral solid dosage formulations described herein may be administered four times a day, three times a day, twice daily, or only once daily.

The sustained release formulations of opioids are administered in an amount sufficient to alleviate pain for an extended period of time, for example about 8 hours to about 24 hours, or for a period of about 12 hours to about 24 hours. The opioid sustained release oral solid dosage formulations described herein may be administered four times a day, three times a day, twice daily, or only once daily.

A therapeutically effective amount of a drug is an amount sufficient to eliminate or to alleviate symptoms of a condition (e.g., reduce the pain compared to the pain present prior to administration of the opioid sustained release formulation).

The drug can be present in the composition in an amount of about 0.5 milligrams to about 1000 milligrams, in an amount of about 1 milligram to about 800 milligrams, in an amount of about 1 milligram to about 200 milligrams, or in an amount of about 1 milligram to about 100 milligrams.

### 4.2. Particle Size Effects on Robustness of Sustained Release Formulations

It has been unexpectedly discovered that the particle size of hydrophilic gums, e.g., xanthan gum, affects dissolution properties of the sustained release formulations and solid dosage forms comprising the sustained release formulations, thereby affecting their robustness. Discovering such a quality-by-design principle and understanding how it applies to the dissolution profile of an extended release formulation of a drug (e.g., an opioid) had heretofore been unknown.

In particular, particle size of hydrophilic gums has been found to affect robustness of ethanol/ethylcellulose granulated formulation. For example, ethanol/ethylcellulose granulated formulations comprising xanthan gum as the hydrophilic gum are robust when the fraction of particles smaller than 53 microns in diameter is about 30% or more. For a different hydrophilic gum, this fraction might be smaller or larger, for example between about 20-80%, about 40-60%, or about 50%. Furthermore, if hydrophilic gum particles are screened through a different mesh filter, the size distribution of the hydrophilic gum required to produce a robust sustained release formulation can be different. Robustness of the sustained release formulations described herein is likely to be a combination of the choice of hydrophilic gum and particle size distribution. In general, the coarser the hydrophilic gum is, the larger the fraction of small particles is required for a robust formulation. Similarly, the finer the hydrophilic gum is, the smaller the fraction of small particles is required for a robust formulation. In some instances, it may be desirable for the formulation to have a percentage of the hydrophilic gum larger than the amount that makes the formulation robust. If the hydrophilic gum is xanthan gum, the formulation may comprise more than 30% of xanthan gum particles smaller than 53 microns, for example, about 40%, about 50%, or about 60%.

Without intending to be bound by any theory, the hydrophilic properties of certain hydrophilic gums (e.g., xanthan gum) contribute to the initial hydration of the sustained release formulations and the solid dosage forms, which in one embodiment comprise a drug, one or more heteropolysaccharide gums and one or more homopolysaccharide gums, and in another embodiment comprise a drug, one or more heteropolysaccharide gums and one or more cross-linking compound selected from monovalent cations, multivalent cations, and salts.

Integrity of sustained release formulations and solid dosage forms comprising hydrophilic gums, e.g., xanthan gum, has also been found to be sensitive to the method used for granulation of formulations comprising xanthan gum particles.

When the granulation method of choice is wet-granulation with non-aqueous solvents such as alcohols, glycerol, propylene glycol, or other non-aqueous solvents, the particle size of xanthan gum will have a substantial effect on hydration and integrity of the granulated sustained release formulation and the solid dosage form.

Rapid hydration of xanthan gum in cold water contributes to the integrity of non-water granulated sustained release formulations and finished solid dosage forms described herein. The rate of hydration of xanthan gum was found to depend on the xanthan gum particle size. Xanthan gum particles of small diameter will, for example, hydrate faster than xanthan gum particles of large diameter. Therefore, non-water granulated sustained release formulations and solid dosage forms comprising xanthan gum particles of smaller average and/or mean diameter will hydrate faster and be more robust than granulated sustained release formulations and solid dosage forms comprising xanthan gum particles of larger average and/or mean diameter.

In some embodiments, wet-granulation with non-aqueous solvents includes a dispersion of one or more hydrophobic materials (e.g., an alkylcellulose, a copolymer of acrylic and methacrylic acid esters, waxes, shellac, zein, hydrogenated vegetable oils, and mixtures of any of the foregoing) in an amount effective to slow the hydration of the formulation when exposed to an environmental fluid.

For example, when the granulation method of choice is wet granulation with ethanol and ethylcellulose, the size of xanthan gum particles affects the hydration properties and integrity of the granulated sustained release formulation and the solid dosage form.

When the granulation method of choice is wet granulation with water or any other aqueous solution, the hydration will be effected using the water from the aqueous solution, and the particle size of xanthan gum will have a lesser, negligible, or even non-existent effect on the hydration of the solid dosage formulation. Based on their poor cold-water solubility, certain homopolysaccharide gums, such as locust bean gum, are not expected to contribute to the initial hydration of the sustained release formulation and solid dosage form. Therefore, the average and/or mean particle size of these homopolysaccharides gums does not affect the hydration properties and integrity of the sustained release formulation and the solid dosage form.

Particle size can be measured using any suitable method used in the art. Perhaps the most common method of measuring particle size comprises screening particles through a sieve. Other exemplary methods include optical methods, e.g., laser diffraction measurements, light microscopy, surface area measurements (e.g., mercury porosimetry, nitrogen gas adsorption, krypton gas adsorption). Other physical measurements can also be used to calculate particle size.

Robustness and integrity of solid dosage forms, such as tablets, capsules, granules and powders, can be measured using several techniques, such as dissolution profile measurements. Exemplary dissolution profile measurements include drug release measurements using a USP Type I, Type II, Type III, or Type IV dissolution apparatus.

### 4.3. Ethanol Effects on Robustness of Sustained Release Formulations

It has been discovered that the sustained release formulations described herein retain their sustained release dissolution properties in the presence of ethanol.

Without intending to be bound by any theory, the physicochemical properties of the hydrophilic compound (e.g., xanthan gum) cross-linked by a cross-linking agent (e.g., locust bean gum), are such that they together form a gum or gum-like matrix, which is insoluble or substantially insoluble in ethanol. These solubility properties of the formulation may be attributed to the hydrophilic nature of the sustained release delivery system, which in one embodiment comprises one or more hydrophilic gums and one or more homopolysaccharide gums, and in another embodiment comprises or one or more hydrophilic gums, and one or more monovalent cations, multivalent cations, and/or salts. Small amounts of hydrophobic agents (e.g., hydrophobic polymers such as ethylcellulose), do not substantially modify the dissolution properties of the formulation in ethanol, presumably because the sustained release delivery system retains its hydrophilic character. Properties of the drug are not likely to affect the gum or gum-like properties of the matrix, making the formulations described herein suitable and/or adaptable to a wide range of drugs.

Several factors are believed to affect the release of a drug from the formulation in the presence of ethanol: solubility of the drug in ethanol, materials comprising the formulation (e.g., hydrophilic compounds are more resistant to ethanol than hydrophobic compounds), and dosage form of the formulation (e.g., tablets are more resistant to ethanol than capsules).

Additional factors believed to affect the release of a drug from the formulation in the presence of ethanol are: degree of compression of the dosage (e.g., harder tablets are more resistant to ethanol than softer tablets), tablet composition (e.g., monolithic tablet compositions are less resistant to ethanol than multiparticulate particle unit dosage forms enclosed in a gelatin capsule), and presence of a gel-like coating which is resistant to dissolution in ethanol (e.g., certain celluloses).

The sustained release formulations described herein can, therefore, be used to prevent or substantially reduce any undesired effects of ethanol on the release of the drug from a formulation. Exemplary undesired effects include dose dumping and altered sustained release dissolution profiles.

Alteration of a sustained release profile can be exhibited, for example, in the bioavailability profile of the drug, such as altered blood plasma concentration time curve after administration of the drug with or without a beverage containing ethanol. Typical parameters measured are the high peak drug concentration (Cₘₐₓ), an increase of which can increase the safety risk of a drug, drug concentration at the end of the therapeutic period (Cₘᵢₙ), a decrease of which can reduce the efficacy of the drug. The sustained release formulations described herein exhibit mean increases in Cₘₐₓ of about 1.7 fold when taken with 40% alcohol compared to 0% alcohol. This is considered acceptable because Cₘₐₓ ratios in an individual when a drug is administered to a fed (with a standard high-fat meal) vs. a fasted individual can vary from about 0.7 to about 3.5, with a mean Cₘₐₓ ratio of about 1.5. Therefore, taking a drug with 40% ethanol has a comparable effect to taking the drug after a high-fat meal. Taking the drug with 20% or 4% ethanol has a smaller effect on Cₘₐₓ than a high-fat meal, as exhibited by the mean Cₘₐₓ ratios of about 1.2 and about 1.1, respectively.

In an exemplary scenario, a formulation with an altered sustained release profile by ethanol may, for example, release a larger amount of the drug shortly after administration (e.g., within 0-6 hours), resulting in a higher-than-intended Cₘₐₓ. If the drug is toxic, a higher-than-intended Cₘₐₓ can lead to harmful side effects for the patient, including death. As a consequence of this rapid release, less drug is available for subsequent release, resulting in a lower-than-intended Cₘᵢₙ at the end of the therapeutic period (i.e., just prior to administration of a subsequent dose). A lower-than-intended Cₘᵢₙ can result in reduced efficacy or even inefficacy of the drug, which can result in recurrence of a condition in a patient.

A higher-than-intended peak drug concentration Cₘₐₓ can be, for example, a concentration more than four times higher than intended Cₘₐₓ. A lower-than-intended Cₘᵢₙ concentration can be, for example, a concentration less than one third of the intended Cₘᵢₙ.

At the Pharmaceutical Sciences Advisory Committee Meeting of Oct. 26, 2005, FDA personnel presented results of a post-approval in vivo study of a known drug. The study showed that taking the drug with a beverage containing 40% alcohol led to a five-fold increase in Cₘₐₓ and taking the same drug with a beverage containing 20% alcohol led to a doubling of Cₘₐₓ. Taking the drug with a beverage containing 5% alcohol led to a small mean effect, but at least one subject doubled their Cₘₐₓ.

The sustained release formulations described herein can, therefore, be used to increase safety of drugs with potentially harmful effects at high concentrations and to reduce abuse of drugs producing a euphoric effect, such as opioids. The formulations described herein can also be used to reduce or prevent harm to a patient in situations where a reduced level of a drug (e.g., lower than the therapeutically beneficial level) can adversely affect the health of the patient. The formulations described herein can be useful for formulation of narrow therapeutic range drugs, sometimes referred to as narrow therapeutic index drugs.

If a formulation described herein is ingested with an alcoholic beverage, or ingested by a patient prior to or after consumption of an alcoholic beverage, the formulation will essentially retain its sustained release properties and will slowly release the drug from the resulting hydrophilic gel matrix.

Because the formulations described herein do not dose dump in the presence of ethanol, they can be used for formulation of drugs that are at risk to be taken with ethanol, such as abuse-potential drugs and drugs prescribed to alcohol and/or drug abusers, or drugs that produce harmful or lethal side effects if over-dosed. Examples of such drugs include opioids.

In addition, patients being treated for conditions such as panic disorder (with or without agoraphobia), bipolar disorder (manic depressive illness), acute manic or mixed episodes associated with bipolar disorder, epilepsy, migraine, attention deficit hyperactivity disorder (ADHD), depression and/or pain may be more likely to consume alcohol compared to the general population. This could be a result of the patients' desire to experience the euphoric effects from inebriation and/or to eliminate or alleviate the symptoms of their condition, such as pain.

Due to the slow release of the drug from the formulations described herein, the patient (e.g., a drug addict) would not experience the euphoria that would be immediately available by abusing conventional formulations (e.g., opioid formulations) by oral inhalation/ingestion or oral ingestion with an alcoholic beverage. Accordingly, the drug formulations described herein would not be abused by patients or their potential for abuse would be significantly reduced (e.g., when compared to conventional opioid formulations).

For example, the sustained release formulations described herein resist extraction of the drug from the formulation by grounding up the solid dosage forms into powder, pouring over 95% ethanol, diluting the resulting solution with water to beverage-strength ethanol, and removing the undissolved material by filtration through a coffee or other paper filter. Ethanol content of hard liquors is typically in the range of 40-45%. This method of extraction is envisioned to be employed by drug addicts, wanting to abuse a drug from the sustained release formulation, such as an opioid, by injecting themselves with the drug extracted from the formulation.

Additionally, because the drug is released slowly from a sustained release formulation over an extended period of time, many sustained release formulations contain relatively high amounts of the drug. Sustained release formulations containing high amounts of drugs can be more harmful to a patient when they fail compared to immediate release formulations, which generally contain smaller amounts of the drug. Therefore, the drug formulations described herein can increase safety of drugs that can be harmful and/or lethal at higher than therapeutically beneficial levels.

### 4.4. Sustained Release Delivery System

The sustained release delivery system comprises at least one hydrophilic compound. In some embodiments, the hydrophilic compound is a gum, for example a heteropolysaccharide gum, forms a gel matrix that releases the drug at a sustained rate upon exposure to liquids.

The rate of release of the drug from the gel matrix depends on the drug's partition coefficient between the components of the gel matrix and the aqueous phase within the gastrointestinal tract. In the compositions described herein, the weight ratio of drug to hydrophilic compound is generally in the range of about 1:0.5 to about 1:25, or in the range of about 1:0.5 to about 1:20. The sustained release delivery system generally comprises the hydrophilic compound in an amount of about 20% to about 80% by weight, in an amount of about 20% to about 60% by weight, in an amount of about 40% to about 60% by weight, or in an amount of about 50% by weight.

The hydrophilic compound can be any known in the art. Exemplary hydrophilic compounds include gums, cellulose ethers, acrylic resins, polyvinyl pyrrolidone, protein-derived compounds, and mixtures thereof. Exemplary gums include heteropolysaccharide gums and homopolysaccharide gums, such as xanthan, tragacanth, pectins, acacia, karaya, alginates, agar, guar, hydroxypropyl guar, carrageenan, locust bean gums, and gellan gums. Exemplary cellulose ethers include hydroxyalkyl celluloses and carboxyalkyl celluloses, such as hydroxyethyl celluloses, hydroxypropyl celluloses, hydroxypropylmethyl-celluloses, carboxy methylcelluloses, and mixtures thereof. Exemplary acrylic resins include polymers and copolymers of acrylic acid, methacrylic acid, methyl acrylate and methyl methacrylate. In some embodiments, the hydrophilic compound is a gum, for example a heteropolysaccharide gum, such as a xanthan gum or derivative thereof. Derivatives of xanthan gum include, for example, deacylated xanthan gum, the carboxymethyl esters of xanthan gum, and the propylene glycol esters of xanthan gum.

In another embodiment, the sustained release delivery system further comprises at least one cross-linking agent. The cross-linking agent can be a compound that is capable of cross-linking the hydrophilic compound to form a gel matrix in the presence of liquids. The sustained release delivery system generally comprises the cross-linking agent in an amount of about 0.5% to about 80% by weight, in an amount of about 2% to about 54% by weight, in an amount of about 20% to about 30% by weight, or in an amount of about 25% by weight.

Exemplary cross-linking agents include homopolysaccharides. Exemplary homopolysaccharides include galactomannan gums, such as guar gum, hydroxypropyl guar gum, and locust bean gum. In some embodiments, the cross-linking agent is a locust bean gum, a guar gum, or a derivative thereof. In other embodiments, the cross-linking agent is an alginic acid derivative or a hydrocolloid.

When the sustained release delivery system comprises at least one hydrophilic compound and at least one cross-linking agent, the ratio of hydrophilic compound to cross-linking agent is generally from about 1:9 to about 9:1, or from about 1:3 to about 3:1.

In some embodiments, the sustained release delivery system comprises one or more cationic cross-linking compounds. In some embodiments, the cationic cross-linking compound can be used instead of or in addition to the cross-linking agent. The cationic cross-linking compound can be used in an amount sufficient to cross-link the hydrophilic compound to form a gel matrix in the presence of liquids. The cationic cross-linking compound is present in the sustained release delivery system in an amount of about 0.5% to about 30% by weight, or from about 5% to about 20% by weight.

Exemplary cationic cross-linking compounds include monovalent metal cations, multivalent metal cations, and inorganic salts, including alkali metal and/or alkaline earth metal sulfates, chlorides, borates, bromides, citrates, acetates, lactates, and mixtures thereof. For example, the cationic cross-linking compound can be one or more of calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride, or mixtures thereof.

When the sustained release delivery system comprises at least one hydrophilic compound and at least one cationic cross-linking compound, the ratio of the hydrophilic compound to the cationic cross-linking compound is generally from about 1:9 to about 9:1, or from about 1:3 to about 3:1.

Two properties of compounds (e.g., the at least one hydrophilic compound and the at least one cross-linking agent; or the at least one hydrophilic compound and the at least one cationic cross-linking compound) that form a gel matrix upon exposure to liquids are fast hydration of the compounds/agents and a gel matrix having a high gel strength. These two properties, which are needed to achieve a slow release gel matrix, are maximized by the particular combination of compounds (e.g., the at least one hydrophilic compound and the at least one cross-linking agent; or the at least one hydrophilic compound and the at least one cationic cross-linking compound). For example, hydrophilic compounds (e.g., xanthan gum) have excellent water-wicking properties that provide fast hydration. The combination of hydrophilic compounds with materials that are capable of cross-linking the rigid helical ordered structure of the hydrophilic compound (e.g., cross-linking agents and/or cationic cross-linking compounds) thereby act synergistically to provide a higher than expected viscosity (i.e., high gel strength) of the gel matrix.

In some embodiments, the sustained release delivery system further comprises one or more pharmaceutical diluents known in the art. Exemplary pharmaceutical diluents include monosaccharides, disaccharides, polyhydric alcohols and mixtures thereof, such as starch, lactose, dextrose, sucrose, microcrystalline cellulose, sorbitol, xylitol, fructose, and mixtures thereof. In other embodiments, the pharmaceutical diluent is water-soluble, such as lactose, dextrose, sucrose, or mixtures thereof. The ratio of pharmaceutical diluent to hydrophilic compound is generally from about 1:8 to about 8:1, or from about 1:3 to about 3:1. The sustained release delivery system generally comprises one or more pharmaceutical diluents in an amount of about 20% to about 80% by weight, for example about 35% by weight. In other embodiments, the sustained release delivery system comprises one or more pharmaceutical diluents in an amount of about 40% to about 80% by weight.

In some embodiments, the sustained release delivery system further comprises one or more hydrophobic polymers. The hydrophobic polymers can be used in an amount sufficient to slow the hydration of the hydrophilic compound without disrupting it. For example, the hydrophobic polymer may be present in the sustained release delivery system in an amount of about 0.5% to about 20% by weight, in an amount of about 2% to about 10% by weight, in an amount of about 3% to about 7% by weight, or in an amount of about 5% by weight.

Exemplary hydrophobic polymers include alkyl celluloses (e.g., C₁₋₆ alkyl celluloses, carboxymethylcellulose), other hydrophobic cellulosic materials or compounds (e.g., cellulose acetate phthalate, hydroxypropylmethylcellulose phthalate), polyvinyl acetate polymers (e.g., polyvinyl acetate phthalate), polymers or copolymers derived from acrylic and/or methacrylic acid esters, zein, waxes, shellac, hydrogenated vegetable oils, and mixtures thereof. The hydrophobic polymer can be, for example, methyl cellulose, ethyl cellulose, or propyl cellulose.

The compositions described herein may be further admixed with one or more wetting agents (such as polyethoxylated castor oil, polyethoxylated hydrogenated castor oil, polyethoxylated fatty acid from castor oil, polyethoxylated fatty acid from hydrogenated castor oil), one or more lubricants (such as magnesium stearate), one or more buffering agents, one or more colorants, and/or other conventional ingredients.

In some embodiments, the robust sustained release formulations comprising a drug are solid dosage formulations, such as orally administrable solid dosage formulations, for example, tablets, capsules comprising a plurality of granules, sublingual tablets, powders, or granules. In some embodiments, the orally administrable solid dosage formulations are tablets. The tablets optionally comprise an enteric coating or a hydrophobic coating.

### 4.5. Robust Sustained Release Formulations Comprising Oxymorphone

In one embodiment, the robust sustained release formulations described herein comprise an analgesically effective amount of oxymorphone or a pharmaceutically acceptable salt thereof.

Administration of oxymorphone is frequently hindered by the very low bioavailability of the oral immediate release formulations of oxymorphone, which require a 4 hourly dosing frequency. The bioavailability of the robust sustained release formulations described herein is sufficiently high that the robust sustained release formulations can be used to treat patients suffering from pain with only once or twice daily dosing.

The robust sustained release formulations of oxymorphone are administered in an amount sufficient to alleviate pain for an extended period of time, for example, for a period of about 8 hours to about 24 hours, or for a period of about 12 hours to about 24 hours.

The oxymorphone sustained release oral solid dosage formulations described herein can be administered four times a day, three times a day, twice daily, or once daily.

In certain embodiments, upon oral ingestion of the robust sustained release formulation comprising oxymorphone and contact of this formulation with gastrointestinal fluids, the robust sustained release formulation swells and gels to form a hydrophilic gel matrix from which the oxymorphone is released. The swelling of the gel matrix causes a reduction in the bulk density of the formulation and provides the buoyancy necessary to allow the gel matrix to float on the stomach contents to provide a slow delivery of the oxymorphone. The hydrophilic matrix, the size of which is dependent upon the size of the original formulation, can swell considerably and become obstructed near the opening of the pylorus. Because the oxymorphone is dispersed throughout the formulation (and consequently throughout the gel matrix), a constant amount of oxymorphone is released per unit time in vivo by dispersion or erosion of the outer portions of the hydrophilic gel matrix. The process continues, with the gel matrix remaining buoyant in the stomach, until substantially all of the oxymorphone is released.

In certain embodiments, the chemistry of certain of the components of the formulation, such as the hydrophilic compound (e.g., xanthan gum), is such that the components are considered to be self-buffering agents which are substantially insensitive to the solubility of the oxymorphone and the pH changes along the length of the gastrointestinal tract. Moreover, the chemistry of the components is believed to be similar to certain known muco-adhesive substances, such as polycarbophil. Muco-adhesive properties are desirable for buccal delivery systems. Thus, the robust sustained release formulation can loosely interact with the mucin in the gastrointestinal tract and thereby provide another mode by which a constant rate of delivery of the oxymorphone is achieved.

In one embodiment, when measured by USP Procedure Drug Release USP 23 (incorporated by reference herein in its entirety), the robust sustained release formulations described herein exhibit an in vitro dissolution rate of about 15% to about 50% by weight oxymorphone after 1 hour, about 45% to about 80% by weight oxymorphone after 4 hours, and at least about 80% by weight oxymorphone after 10 hours. The in vitro and in vivo release characteristics of the robust sustained release formulations described herein can be modified using mixtures of one or more different water insoluble and/or water soluble compounds, using different plasticizers, varying the thickness of the sustained release film, including providing release-modifying compounds in the coating, and/or by providing passageways through the coating.

Some embodiments provide robust sustained release solid dosage formulations comprising from about 1 mg to about 200 mg of oxymorphone hydrochloride, or from about 5 mg to about 80 mg of oxymorphone hydrochloride; and from about 80 mg to about 200 mg of a sustained release delivery system, or from about 120 mg to about 200 mg of a sustained release delivery system, or about 160 mg of a sustained release delivery system; where the sustained release delivery system comprises about 8.3 to about 41.7% locust bean gum, or about 25% locust bean gum; from about 8.3 to about 41.7% xanthan gum having at least about 30% of particles smaller than about 53 microns in diameter, or about 25% xanthan gum with at least about 30% of particles smaller than about 53 microns in diameter; from about 20 to about 55% dextrose, or about 35% dextrose; from about 5 to about 20% calcium sulfate dihydrate, or about 10% calcium sulfate dihydrate; and from about 2 to 10% ethyl cellulose, or about 5% ethyl cellulose.

Other embodiments provide robust sustained release solid dosage formulations comprising from about 1 mg to about 200 mg of oxymorphone hydrochloride, or from about 5 mg to about 80 mg of oxymorphone hydrochloride; and from about 80 mg to about 200 mg of a sustained release delivery system, or from about 120 mg to about 200 mg of a sustained release delivery system, or about 160 mg of a sustained release delivery system; where the sustained release delivery system comprises from about 8.3 to about 41.7% locust bean gum, or about 25% locust bean gum; from about 8.3 to about 41.7% xanthan gum wherein at least about 30% of the xanthan gum particles can pass through a #270 mesh sieve, or about 25% xanthan gum of which at least about 30% of the particles can pass through a #270 mesh sieve; from about 20 to about 55% dextrose, or about 35% dextrose; from about 5 to about 20% calcium sulfate dihydrate, or about 10% calcium sulfate dihydrate; and from about 2 to about 10% ethyl cellulose, or about 5% ethyl cellulose.

Some embodiments provide robust sustained release solid dosage formulations comprising from about 1 mg to about 200 mg of oxymorphone hydrochloride, or from about 5 mg to about 80 mg of oxymorphone hydrochloride; and from about 200 mg to about 420 mg of a sustained release delivery system, or from about 300 mg to about 420 mg of a sustained release delivery system, or about 360 mg of a sustained release delivery system; where the sustained release delivery system comprises from about 8.3 to about 41.7% locust bean gum, or about 25% locust bean gum; from about 8.3 to about 41.7% xanthan gum having at least about 30% of particles smaller than about 53 microns in diameter, or about 25% xanthan gum with at least about 30% of particles smaller than about 53 microns in diameter; from about 20 to about 55% dextrose, or about 35% dextrose; from about 5 to about 20% calcium sulfate dihydrate, or about 10% calcium sulfate dihydrate; and from about 2 to 10% ethyl cellulose, or about 5% ethyl cellulose.

Other embodiments provide robust sustained release solid dosage formulations comprising from about 1 mg to about 200 mg of oxymorphone hydrochloride, or from about 5 mg to about 80 mg of oxymorphone hydrochloride; and from about 200 mg to about 420 mg of a sustained release delivery system, or from about 300 mg to about 420 mg of a sustained release delivery system, or about 360 mg of a sustained release delivery system; where the sustained release delivery system comprises from about 8.3 to about 41.7% locust bean gum, or about 25% locust bean gum; from about 8.3 to about 41.7% xanthan gum wherein at least about 30% of the xanthan gum particles can pass through a #270 mesh sieve, or about 25% xanthan gum of which at least about 30% of the particles can pass through a #270 mesh sieve; from about 20 to about 55% dextrose, or about 35% dextrose; from about 5 to about 20% calcium sulfate dihydrate, or about 10% calcium sulfate dihydrate; and from about 2 to 10% ethyl cellulose, or about 5% ethyl cellulose.

When administered orally to patients the robust sustained release formulations described herein exhibit the following in vivo characteristics: (a) a peak plasma level of oxymorphone occurs within about 2 to about 6 hours after administration; (b) the duration of the oxymorphone analgesic effect is about 8 to about 24 hours; and (c) the relative oxymorphone bioavailability is about 0.5 to about 1.5 compared to an orally administered aqueous solution of oxymorphone.

While the oxymorphone compositions described herein can be administered as the sole active pharmaceutical compound in the methods described herein, they can also be used in combination with one or more compounds which are known to be therapeutically effective against pain.

In one embodiment, pharmaceutical kits comprising one or more containers filled with one or more of robust sustained release oxymorphone formulations described herein are provided. The kits can further comprise other pharmaceutical compounds known in the art to be therapeutically effective against pain, and instructions for use.

### 4.6. Preparation of the Robust Sustained Release Formulations

The robust sustained release formulations described herein can be prepared by wet granulation methods. The solid dosage forms described herein can be prepared by direct compression or by wet granulation of the formulations.

In some embodiments, the sustained release formulations are manufactured by a wet granulation technique. In the wet granulation technique, the components (e.g., hydrophilic compounds such a xanthan gum, cross-linking agents, pharmaceutical diluents, cationic cross-linking compounds, hydrophobic polymers, etc.) are mixed together and then moistened with one or more liquids (e.g., water, propylene glycol, glycerol, alcohol) to produce a moistened mass that is subsequently dried. The dried mass is then milled with conventional equipment into granules of the sustained release delivery system. Thereafter, the sustained release delivery system is mixed in the desired amounts with the drug and, optionally, one or more wetting agents, one or more lubricants, one or more buffering agents, one or more coloring agents, or other conventional ingredients, to produce a granulated composition. The sustained release delivery system and the drug can be blended with, for example, a high shear mixer. The drug can be finely and homogeneously dispersed in the sustained release delivery system. The granulated composition, in an amount sufficient to make a uniform batch of tablets, is subjected to tableting in a conventional production scale tableting machine at normal compression pressures, i.e., about 2,000-16,000 psi. The mixture should not be compressed to a point where there is subsequent difficulty with hydration upon exposure to liquids. Exemplary methods for preparing sustained release delivery systems are described in U.S. Pat. Nos. 4,994,276, 5,128,143, 5,135,757, 5,455,046, 5,512,297 and 5,554,387, the disclosures of which are incorporated by reference herein in their entirety.

It has been unexpectedly discovered that the particle size of the hydrophilic compound (e.g., xanthan gum) affects the robustness and integrity of the formulation and solid dosage forms when the sustained release delivery system is wet-granulated with a non-aqueous solution, such as an ethanol/ethylcellose suspension.

In particular, the fraction of small particles (e.g., smaller than 53 microns in diameter) of the hydrophilic compound (e.g., xanthan gum) affects the robustness and integrity of the sustained release formulations and solid dosage forms prepared by wet-granulation with a non-aqueous solvent. For example, if the xanthan gum used to make the formulation contains less than a certain fraction (e.g., about 30%) of small xanthan gum particles, the sustained release formulation is prone to failure. When the fraction of small xanthan gum particles used to make the formulation meets or exceeds certain threshold value, the formulations are robust and not prone to failure. For example, once a threshold fraction of about 30% of xanthan gum particles smaller than 53 microns in diameter is met or exceeded, no change in robustness and integrity of the formulation and solid dosage form is observed (see Table 4).

It will be apparent to one skilled in the art that other combinations of xanthan gum particle sizes and threshold fractions may also be used to manufacture robust sustained release formulations described herein. For example, a formulation comprising xanthan gum particles smaller than 45, 38, 32, 25, or 20 microns in diameter may be robust when the threshold fraction is less than about 30%, for example between about 5-25%, or between about 10-20%. A formulation comprising xanthan gum particles smaller than 63, 75, 90, 106, 125, or 150 microns in diameter may be robust when the threshold fraction is more than about 30%, for example between about 30-100%, or between about 50-90%. Robustness and integrity of sustained release formulations and solid dosage forms granulated with a non-aqueous solution can be improved by controlling the particle size distribution of the hydrophilic compound (e.g., xanthan gum). Control of the particle size distribution of the hydrophilic compound can be achieved, for example, by screening the hydrophilic compound (e.g., xanthan gum) particles through a sieve, (e.g., a #270 mesh sieve) which allows particles smaller than a certain size (e.g., 53 microns in diameter) to pass through. Batches, lots, and combinations thereof having a desired fraction of particles of a desired size can then be used for combination with other components to make a robust sustained release formulation.

Alternatively, the hydrophilic compound (e.g., xanthan gum) can be manufactured to have a desired particle distribution, in which case no screening or other processing is required. Furthermore, the hydrophilic compound having a desired particle size distribution (such as average particle size, mean particle size, minimum particle size, maximum particle size, or a combination thereof) can be received from an external source, for example, a commercial manufacturer or a distributor.

When the sustained release delivery system is wet-granulated with water or any other aqueous solution, the particle size of the hydrophilic compound (e.g., xanthan gum) does not appear to affect the robustness and integrity of the sustained release formulation and the solid dosage form (see Table 5).

The average particle size of the pharmaceutical formulations before tableting is from about 50 microns to about 400 microns, or from about 185 microns to about 265 microns. The average density of the pharmaceutical formulations is from about 0.3 g/ml to about 0.8 g/ml, or from about 0.5 g/ml to about 0.7 g/ml. The tablets formed from the pharmaceutical formulations are generally from about 6 to about 8 kg hardness.

When the tableting step in making the solid dosage formulation is performed using wet granulation instead of direct compression, the particle size of the hydrophilic compound (e.g., xanthan gum) does not affect the robustness and dissolution properties of the solid dosage form.

In some embodiments, the sustained release coatings over an inner core comprise at least one drug. For example, the inner core comprising the drug can be coated with a sustained release film, which, upon exposure to liquids, releases the drug from the core at a sustained rate.

In one embodiment, the sustained release coating comprises at least one water insoluble compound. The water insoluble compound can be a hydrophobic polymer. The hydrophobic polymer can be the same as or different from the hydrophobic polymer used in the sustained release delivery system. Exemplary hydrophobic polymers include alkyl celluloses (e.g., C₁₋₆ alkyl celluloses, carboxymethylcellulose), other hydrophobic cellulosic materials or compounds (e.g., cellulose acetate phthalate, hydroxypropylmethylcellulose phthalate), polyvinyl acetate polymers (e.g., polyvinyl acetate phthalate), polymers or copolymers derived from acrylic and/or methacrylic acid esters, zein, waxes (alone or in admixture with fatty alcohols), shellac, hydrogenated vegetable oils, and mixtures thereof. The hydrophobic polymer can be, for example, methyl cellulose, ethyl cellulose, or propyl cellulose. The robust sustained release formulations can be coated with a water insoluble compound to a weight gain from about 1 to about 20% by weight.

The sustained release coating can further comprise at least one plasticizer such as triethyl citrate, dibutyl phthalate, propylene glycol, polyethylene glycol, or mixtures thereof.

The sustained release coating can also contain at least one water soluble compound, such as polyvinylpyrrolidones, hydroxypropylmethylcelluloses, or mixtures thereof. The sustained release coating can comprise at least one water soluble compound in an amount from about 1% to about 6% by weight, for example, in an amount of about 3% by weight.

The sustained release coating can be applied to the drug core by spraying an aqueous dispersion of the water insoluble compound onto the drug core. The drug core can be a granulated composition made, for example, by dry or wet granulation of mixed powders of drug and at least one binding agent; by coating an inert bead with an drug and at least one binding agent; or by spheronizing mixed powders of an drug and at least one spheronizing agent. Exemplary binding agents include hydroxypropylmethylcelluloses. Exemplary spheronizing agents include microcrystalline celluloses. The inner core can be a tablet made by compressing the granules or by compressing a powder comprising a drug.

In other embodiments, the compositions comprising at least one drug and a sustained release delivery system, as described herein, are coated with a sustained release coating, as described herein. In still other embodiments, the compositions comprising at least one drug and a sustained release delivery system, as described herein, are coated with a hydrophobic polymer, as described herein. In still other embodiments, the compositions comprising at least one drug and a sustained release delivery system, as described herein, are coated with an enteric coating, such as cellulose acetate phthalate, hydroxypropylmethylcellulose phthalate, polyvinylacetate phthalate, methacrylic acid copolymer, shellac, hydroxypropylmethylcellulose succinate, cellulose acetate trimelliate, or mixtures thereof. In still other embodiments, the compositions comprising at least one drug and a sustained release delivery system, as described herein, are coated with a hydrophobic polymer, as described herein, and further coated with an enteric coating, as described herein. In any of the embodiments described herein, the compositions comprising the drug and a sustained release delivery system, as described herein, can optionally be coated with a hydrophilic coating which may be applied above or beneath the sustained release film, above or beneath the hydrophobic coating, and/or above or beneath the enteric coating. Exemplary hydrophilic coatings comprise hydroxypropylmethylcellulose.

Without intending to be bound by any theory of the invention, upon oral ingestion of the drug sustained release formulation and contact of the formulation with gastrointestinal fluids, the sustained release formulation swells and gels to form a hydrophilic gel matrix from which the drug is released. The swelling of the gel matrix causes a reduction in the bulk density of the formulation and provides the buoyancy necessary to allow the gel matrix to float on the stomach contents to provide a slow delivery of the drug. The hydrophilic matrix, the size of which is dependent upon the size of the original formulation, can swell considerably and become obstructed near the opening of the pylorus. Because the drug is dispersed throughout the formulation (and consequently throughout the gel matrix), a constant amount of drug can be released per unit time in vivo by dispersion or erosion of the outer portions of the hydrophilic gel matrix. This phenomenon is referred to as a zero order release profile or zero order kinetics. The process continues, with the gel matrix remaining buoyant in the stomach, until substantially all of the drug is released.

Without intending to be bound by any theory of the invention, the chemistry of certain of the components of the formulation, such as the hydrophilic compound (e.g., xanthan gum), is such that the components are considered to be self-buffering agents which are substantially insensitive to the solubility of the drugs and the pH changes along the length of the gastrointestinal tract. Moreover, the chemistry of the components is believed to be similar to certain known muco-adhesive substances, such as polycarbophil. Muco-adhesive properties are desirable for buccal delivery systems. Thus, it may be possible that the sustained release formulation could potentially loosely interact with the mucin in the gastrointestinal tract and thereby provide another mode by which a constant rate of delivery of the drug is achieved.

The two phenomena discussed above (hydrophilic gel matrix and muco-adhesive properties) are possible mechanisms by which the robust sustained release formulations described herein could interact with the mucin and fluids of the gastrointestinal tract and provide a constant rate of delivery of the drugs.

### 4.7. Usefulness of Robust Sustained Release Formulations

The robust sustained release formulations and solid dosage forms described herein are useful for formulation of drugs that pose a risk to the patient in case of a formulation failure. The formulations and solid dosage forms comprising the formulations described herein are useful for providing (e.g., prescribing, administering) drugs that pose a risk to the patient in case of a formulation failure. Examples of such drugs include, for example, opioids such as oxymorphone.

The robust sustained release formulations and solid dosage forms described herein are useful for treating a condition (e.g., pain), by prescribing and/or administering a therapeutically effective amount of the robust sustained release formulations of the drug (e.g., an opioid such as oxymorphone) to a patient who could consume ethanol while being treated with the drug. A therapeutically effective amount is an amount sufficient to eliminate the condition or to alleviate the condition (i.e., reduce the symptoms compared to the symptoms present prior to administration of the robust sustained release formulation).

While the formulations and solid dosage forms described herein can be administered as the sole active pharmaceutical composition in the methods described herein, they can also be used in combination with one or more compounds and/or compositions that are known to be therapeutically effective against the condition.

Pharmaceutical kits comprising one or more of the drug formulations described herein are provided. Pharmaceutical kits can, for example, comprise one or more containers filled with one or more of the robust sustained release formulations and/or solid dosage forms described herein. The kits can further comprise other pharmaceutical compounds known in the art to be therapeutically effective against a condition, and instructions for use.

### 5. EXAMPLES

The following examples are for purposes of illustration only and are not intended to limit the scope of the appended claims.

Some experiments were performed with albuterol sulfate, which has dosage, solubility and other physicochemical properties similar to opioids, such as oxymorphone and oxycodone.

### Example 1

### Preparation of TIMERx-N® Sustained Release Delivery System Using Ethanol/Ethylcellulose Granulation

Lots of TIMERx-N® sustained release delivery system were prepared according to the procedures related to those identified in U.S. Pat. Nos. 4,994,276, 5,128,143 and 5,554,387, incorporated herein by reference in their entirety.

Lots of xanthan gum (Jungbunzlauer, Perhoven, Austria or CP Kelco, Chicago, Ill.) were particle-size tested using a series of mesh sieves. These sieves included a #270 mesh sieve, which allowed particles smaller than 53 microns in diameter to pass through (fine particles). The weight fraction of xanthan gum particles passing through the sieves (i.e., fraction of fine xanthan gum) was determined. Batches with known fractions of fine xanthan gum particles were then prepared. TIMERx-N® was prepared by dry blending the requisite amounts of xanthan gum, locust bean gum, calcium sulfate, and dextrose in a high speed mixer/granulator for 3 minutes. A slurry of hydrophobic polymer (ethylcellulose) was prepared by dissolving ethyl cellulose in ethyl alcohol. The slurry was added to the dry blended mixture and the material was subsequently granulated for 4 minutes while running the choppers/impeller. The granulation was then dried in a fluid bed dryer to a LOD (loss on drying) of less than 9% by weight (e.g., typical LOD was ˜3-5%). The granulation was then milled using a 1.0 mm (0.040″) screen. The ingredients of the sustained release excipient are set forth in Table 1:

TABLE 1
TIMERx-N ® Composition

| Component | % |
|---|---|
| 1. Xanthan Gum | 25 |
| 2. Locust Bean Gum | 25 |
| 3. Calcium Sulfate | 10 |
| 4. Dextrose | 35 |
| 5. Ethyl Cellulose | 5 |
| 6. Ethyl Alcohol | ~20* |

*removed during processing

### Example 2

### Preparation of TIMERx-M50A® Sustained Release Delivery System Using Water Granulation

Lots of TIMERx-M50A® sustained release delivery system were prepared according to the procedures related to those identified in U.S. Pat. No. 5,399,358, incorporated herein by reference in its entirety.

Xanthan gum batches with known fractions of fine particles were prepared according to Example 1. TIMERx-M50® was prepared by dry blending the requisite amounts of xanthan gum, locust bean gum, calcium sulfate, and mannitol in a high speed mixer/granulator for 3 minutes. While running choppers/impellers, water was added to the dry blended mixture, and the mixture was granulated for another 3 minutes. The granulation was then dried in a fluid bed dryer to a loss on drying (LOD) of less than about 6% by weight. Typical LOD was between ˜3-5%. The granulation was then milled using a 0.065″ screen. The ingredients of the sustained release delivery system are set forth in Table 2.

TABLE 2
TIMERx-M50A ® Composition

| Component | % |
|---|---|
| Xanthan Gum | 20 |
| Locust Bean Gum | 30 |
| Mannitol | 40 |
| Calcium Sulfate | 10 |
| Water | ~30–40* |

*removed during processing

### Example 3

### Preparation of Sustained Release Formulations and Solid Dosage Forms with Variable Amounts of Fine Xanthan Gum

A sustained release formulation was prepared by screening albuterol sulfate, ProSolv SMCC® 90 (Silicified Microcrystalline Cellulose, JRS Pharma LP, Patterson, N.Y.) and TIMERx-N®or TIMERx-M50A® separately through a #20 mesh sieve. The albuterol sulfate, ProSolv SMCC® 90 and either TIMERx-N® or TIMERx-M50A®, prepared according to Examples 1 and 2, respectively, were blended for 11 minutes in a Patterson-Kelley P/K Blendmaster V-Blender. Pruv™ (Sodium Stearyl Fumarate, NF, JRS Pharma LP, Patterson, N.Y.) was added to this mixture and the mixture was blended for five minutes. The blended granulation was compressed to 224.0 mg and ˜11 Kp hardness on a tablet press using 5/16″ round standard concave beveled edge tooling. The final tablet composition is listed in the Table 3.

TABLE 3
Tablet Composition

| Component | % | mg/tablet |
|---|---|---|
| Albuterol sulfate | 17.9 | 40.0 |
| TIMERx-N ® or TIMERx-M50A ® | 71.4 | 160.0 |
| ProSolv SMCC ® 90 | 8.9 | 20.0 |
| Pruv ™ | 1.8 | 4.0 |

### Example 4

### Dissolution Profile Measurements of Solid Dosage Forms with Variable Amounts of Fine Xanthan Gum

Albuterol sulfate tablets with TIMERx-N® and TIMERx-M50A® sustained release delivery systems were prepared as described in Example 3. Dissolution profiles of tablets were evaluated using a USP Apparatus 2 dissolution tester in 900 mL of 50 mM potassium phosphate buffer (pH 4.5). The solution was stirred at 50 r.p.m. A series of samples of about 1.5 mL were withdrawn at predetermined intervals for a period of up to 14 hours.

Drug release for all tablets was monitored by RP-HPLC using a Waters Symmetry® C18 column (4.6×250 mm) (or equivalent) preceded by a Phenomenex® SecurityGuard™ C18 (4×3.0 mm) guard column. Monitoring wavelength was set to 226 nm. The mobile phase consisted of buffer: acetonitrile:methanol in 85:10:5 v/v ratios. The buffer consisted of 1 mL triethylamine and 1 mL trifluoroacetic acid in 1 L of H₂O. The column temperature was 30° C. and the flow rate was set to 1.5 mL/min. To determine the percentage of drug released at each timepoint, the concentration of the sample taken at that timepoint was compared to the concentration of a standard solution. The standard solution was prepared by dissolving 45 mg of albuterol sulfate in 100 mL of 50 mM potassium phosphate buffer (pH 4.5) and then taking 5 mL of this solution and diluting it to 50 mL with more of 50 mM potassium phosphate buffer (pH 4.5).

Results of dissolution experiments with tablets made with alcohol/ethylcellulose-granulated TIMERx-N® comprising xanthan gum with different particle size distributions are shown in Table 4.

TABLE 4

| | | Sustained release delivery system | | | | | | |
|---|---|---|---|---|---|---|---|---|
| | | % albuterol sulfate released | | | | | | |
| | | TIMERx-N ® (ethanol/ethylcellulose-granulated) | | | | | | |
| | | Fraction of fine xanthan gum - | | | | | | |
| Time | | 13.7% | 27.9% | 31.6% | 42.0% | 48.5% | 85.2% | 88.8% |
| 0.5 | hr | 102.3 | 94.2 | 17.2 | 17.7 | 16.8 | 19.0 | 18.9 |
| 1 | hr | 102.7 | 96.9 | 28.7 | 27.9 | 27.6 | 29.3 | 29.0 |
| 2 | hrs | | | 45.2 | 43.4 | 44.3 | 44.9 | 44.5 |
| 3 | hrs | | | 57.8 | 55.5 | 57.1 | 56.8 | 56.7 |
| 4 | hrs | | | 68.0 | 65.9 | 67.0 | 66.3 | 66.7 |
| 6 | hrs | | | 82.6 | 79.9 | 80.8 | 79.5 | 80.8 |
| 8 | hrs | | | 91.7 | 88.6 | 89.2 | 88.1 | 89.8 |
| 10 | hrs | | | 97.2 | 93.7 | 94.0 | 93.1 | 94.5 |
| 12 | hrs | | | 100.5 | 96.6 | 96.9 | 96.3 | 97.2 |
| 14 | hrs | | | 102.7 | 97.9 | 98.4 | 98.2 | 98.7 |

Tablets comprising 13.7% and 27.9% of fine xanthan gum in the ethanol/ethylcelluose-granulated TIMERx-N® released nearly the entire quantity of drug almost immediately. This is an example of undesired dose dumping. Tablets with 31.6% or more of fine xanthan gum dissolved in the expected sustained release manner. The data in Table 4 indicate that there appears to be no substantial difference in dissolution profiles of formulations containing between about 31.6% and about 88.8% of fine xanthan gum particles.

Results of dissolution experiments with tablets made with water-granulated TIMERx-M50A® comprising xanthan gum with different particle size distributions are shown in Table 5.

TABLE 5

Sustained release
delivery system
% albuterol sulfate released
TIMERx-M50A ®
(water-granulated)
Xanthan gum
particle size

| | <#80 mesh | <#200 mesh |
|---|---|---|
| Time | (<180 microns) | (<75 microns) |
| 0.5 hr | 17.5 | 19.8 |
| 1 hr | 29.5 | 29.9 |
| 2 hrs | 47.6 | 45.4 |
| 3 hrs | 62.6 | 58.1 |
| 4 hrs | 74.2 | 68.6 |
| 6 hrs | 88.4 | 83.0 |
| 8 hrs | 96.8 | 91.6 |
| 10 hrs | 101.0 | 96.5 |
| 12 hrs | 103.4 | 99.0 |
| 14 hrs | 104.8 | 99.9 |

Tablets made by direct compression of water-granulated TIMERx-M50A® formulations comprising xanthan gum are not sensitive to xanthan gum particle size. The data in Table 5 indicate that there appears to be no substantial difference between the dissolution profiles of tablets made with xanthan gum having particle size of less than 180 microns and less than 75 microns when xanthan gum is granulated with water in the process of making the formulation.

Table 6 shows dissolution profiles of tablets made by direct compression and granulation of ethanol/ethylcellulose-granulated sustained release formulations with different fractions of #270 (fine) mesh xanthan gum particles.

TABLE 6

| | Sustained release | | | |
|---|---|---|---|---|
| | delivery system | | | |
| | % albuterol sulfate released | | | |
| | TIMERx-N ® | | | |
| | (ethanol/ethylcellulose-granulated) | | | |
| | Fraction of fine | | | |
| | xanthan gum | | | |
| | | 27.9% | | |
| | 27.9% | (tablet | 34.8% | 42.0% |
| | (tablet made | made by | (tablet made | (tablet made |
| | by direct | wet | by direct | by direct |
| Time | compression) | granulation) | compression) | compression) |
| 0.5 hr | 80.1 | 17.3 | 17.2 | 17.9 |
| 1 hr | 92.8 | 25.6 | 28.7 | 29.0 |
| 2 hrs | | 39.2 | 45.2 | 46.3 |
| 3 hrs | | 50.7 | 57.8 | 59.7 |
| 4 hrs | | 59.6 | 68.0 | 70.5 |
| 6 hrs | | 72.5 | 82.6 | 83.9 |
| 8 hrs | | 81.2 | 91.7 | 92.1 |
| 10 hrs | | 88.1 | 97.2 | 97.2 |
| 12 hrs | | 91.9 | 100.5 | 99.2 |
| 14 hrs | | | 102.7 | 99.7 |

Comparison of dissolution profiles of tablets comprising TIMERx-N® that were manufactured either using direct compression or wet granulation in the tableting step, shows that robustness of tablets appears to be sensitive to xanthan gun particle size when the tablets are manufactured by direct compression, but not when they are manufactured by wet granulation. Tablets with ethanol/ethylcellulose-granulated TIMERx-N® with 27.9% of fine particles had desired dissolution profiles when tableted using wet granulation, but not when tableted using direct compression. Direct compression of ethanol/ethylcellulose-granulated formulations produced tablets with desired dissolution profiles when the fraction of fine xanthan gum was more than about 30%.

### Example 5

### Ethanol Resistance of Solid Dosage Forms with Variable Amounts of Fine Xanthan Gum

Tablets of TIMERx-N® formulations of albuterol sulfate were prepared as described in Example 3. Dissolution profiles of each formulation were measured as described in Example 4. A medium of 40% ethanol and 60% 0.1 M HCl was used as a model of dissolution in the presence of alcohol. 0.1 M HCl was chosen to mimic the biological environment of upper GI tract/stomach area, where the sustained release formulation first begins to release the drug.

Dissolution experiments were performed using a USP II Type dissolution apparatus according to methods described above. Results of dissolution experiments with tablets made with alcohol/ethylcellulose-granulated TIMERx-N® comprising xanthan gum with different particle size distributions are shown in Table 7.

TABLE 7

Sustained release delivery system
% albuterol sulfate released
TIMERx-N ® (ethanol/ethylcellulose-granulated)
Fraction of fine xanthan gum in dissolution medium

| | | | | 35% | | | | | 86% in |
|---|---|---|---|---|---|---|---|---|---|
| | | 28% in | 28% in | in | 35% in | 42% in | 42% in | 86% in | 40% |
| Time | | buffer | 40% Ethanol | buffer | Ethanol | buffer | Ethanol | buffer | Ethanol |
| 0.5 | hr | 98.5 | 100.0 | 15.7 | 28.8 | 18.7 | 16.1 | 17.8 | 15.8 |
| 1 | hr | 99.9 | 101.2 | 26.8 | 38.1 | 29.6 | 25.5 | 27.5 | 24.1 |
| 2 | hrs | 99.8 | 99.5 | 45.2 | 51.5 | 46.9 | 40.3 | 45.1 | 34.9 |
| 3 | hrs | 99.8 | 99.5 | 58.7 | 63.6 | 60.2 | 53.0 | 57.9 | 44.6 |
| 4 | hrs | 99.8 | 99.5 | 69.6 | 76.9 | 70.9 | 63.7 | 67.7 | 52.5 |
| 6 | hrs | 99.8 | 99.5 | 86.5 | 92.8 | 85.4 | 78.0 | 81.5 | 66.0 |
| 8 | hrs | 99.8 | 99.5 | 96.8 | 99.0 | 94.2 | 87.6 | 89.4 | 74.2 |
| 10 | hrs | 99.8 | 99.5 | 103.3 | 101.7 | 98.9 | 96.6 | 94.3 | 80.9 |
| 12 | hrs | 99.8 | 99.5 | 105.9 | 103.5 | 101.7 | 103.1 | 96.9 | 85.5 |
| 14 | hrs | 99.8 | 99.5 | 108.0 | 105.0 | 103.7 | 106.5 | 98.1 | 88.9 |

Tablets comprising 28% of fine xanthan gum in the ethanol/ethylcelluose-granulated TIMERx-N® released nearly the entire quantity of drug almost immediately. This is an example of undesired dose dumping. Tablets with 35% or more of fine xanthan gum dissolve in the expected sustained release manner. The data in Table 7 indicate that there appears to be no substantial difference in dissolution profiles of formulations containing between about 35% and about 86% of fine xanthan gum particles, although the formulation containing about 86% of fine xanthan gum particles dissolved slightly slower in 40% ethanol solution than in a standard buffer.

Therefore, formulations comprising about 30% or more of fine xanthan gum, exhibit robust dissolution properties, and dissolve in a sustained release manner in the presence and absence of beverage-strength ethanol.

### Example 6

### Preparation of Robust Sustained Release Oxymorphone Formulations and Solid Dosage Forms

A controlled release delivery system was prepared by dry blending xanthan gum, locust bean gum, calcium sulfate dihydrate, and dextrose in a high speed mixed/granulator for a few minutes. A slurry was prepared by mixing ethyl cellulose with alcohol. While running choppers/impellers, the slurry was added to the dry blended mixture, and granulated for a few minutes. The granulation was then dried to a LOD (loss on drying) of less than about 10% by weight. The granulation was then milled using a screen. The relative quantities of the ingredients used to prepare the sustained release delivery system are listed in Table 8A.

TABLE 8A

| Excipient | % of Formulation |
|---|---|
| Locust Bean Gum, FCC | 25.0 |
| Xanthan Gum, NF | 25.0 |
| Dextrose, USP | 35.0 |
| Calcium Sulfate Dihydrate, NF | 10.0 |
| Ethylcellulose, NF | 5.0 |
| Alcohol, SD3A (Anyhdrous) | (10) |
| Total | 100.0 |

Tablets comprising 40 mg of oxymorphone hydrochloride were prepared using the controlled release delivery system shown in Table 8A. The quantities of ingredients per tablet are listed in Table 8B.

TABLE 8B

| | Amount per tablet |
|---|---|
| Component | [mg] |
| Oxymorphone HCl, USP (mg) | 40 |
| TIMERx-N ® sustained release delivery system | 160 |
| Silicified microcrystalline cellulose, N.F. | 20 |
| Sodium stearyl fumarate, N.F. | 2 |
| Total theoretical weight of uncoated drug product | 222 |
| Methylparaben | 0.08140 |
| Opadry (colored) | 8.88 |
| Opadry (clear) | 1.11 |
| Total theoretical weight of final drug product | 232.07 |
| (coated) | |

### Example 7

### Extraction-Resistance of Powdered Sustained Release Oxymorphone Tablets

Tablets of TIMERx-N® sustained release formulations with 40 mg of oxymorphone were tested for abuse potential in an intravenous route of administration. A person, such as a drug addict, trying to abuse the formulation, may attempt to extract the opioid from the tablets and inject themselves with the resulting solution.

Tablets of TIMERx-N® sustained release formulations with 40 mg of oxymorphone were prepared according to procedures in Example 6 and ground into powder. In the water extraction test, the resulting powder was dispersed into 30 mL of water and stirred for 5 seconds. In the 95% ethanol/water extraction test, the resulting powder was dispersed into 15 mL of 95% ethanol, stirred for 5 seconds, and then diluted with an additional 15 mL of water. In the 95% ethanol extraction test, the resulting powder was dispersed into 30 mL of 95% ethanol and stirred for 5 seconds. In each test, the resulting solution was allowed to set for 15 minutes before being filtered through a paper filter. Oxymorphone recovery from the filtered solutions was measured using HPLC at 40° C., using a Zorbax® XDB-C18 column and a UV detector set at 230 nm. Recovery of oxymorphone from each test is shown in Table 9.

TABLE 9

| | % Dose recovered after extraction in | | |
|---|---|---|---|
| Tablet | water | 95% ethanol/water | 95% ethanol |
| 1 | 3.3 | 14.8 | 87.3 |
| 2 | 3.8 | 13.3 | 85.3 |
| 3 | 3.3 | 11.3 | 82.5 |
| Mean | 3.5 | 13.0 | 85.0 |

When sustained release tablets comprising 40 mg of oxymorphone, formulated with TIMERx-N® made with xanthan gum in which at least 30% of particles can pass through a #270 mesh sieve, were powdered and extracted with water, approximately 3-4% of oxymorphone was released into water after 15 minutes. To mimic abuse by dropping a tablet into 95% ethanol and then diluting it to an ingestible concentration, powdered tablets were first suspended in 95% ethanol for 5 seconds, followed by dilution with water to provide a 47.5% ethanol solution. In this experiment, approximately 1-15% of oxymorphone was released into the water/ethanol solution after 15 minutes. The powdered sustained release 40 mg oxymorphone tablets formulated with TIMERx-N® with xanthan gum of which at least 30% of the particles can pass through a #270 mesh sieve, therefore, resist extraction in more than one potential abuse scenario.

### Example 8

### Dissolution Profiles of Sustained Release Oxymorphone Tablets in the Presence of Beverage-Strength Ethanol

Sustained release 40 mg oxymorphone tablets were prepared as described in Example 6. Dissolution tests were performed on sets of 12 tablets in 500 mL of 0.1N HCl and ethanol/0.1N HCl solutions at 4%, 20%, and 40% ethanol concentrations. Oxymorphone release was determined by HPLC as described above.

Tablets remained intact throughout the dissolution tests in all media. Mean concentrations of oxymorphone released are shown in Table 10A. Similarity factors (f₂) for the ethanol dissolution media against the 0.1N HCl medium were calculated using standard methods and the results indicate that the drug release rate is inversely correlated with the amount of ethanol in the dissolution medium (Table 10B). An increase in ethanol content of the dissolution medium moderately decreased the drug release rate.

Results of dissolution experiments are summarized in Table 10A.

TABLE 10A

| | Mean % oxymorphone released (n = 12) | | | | | | |
|---|---|---|---|---|---|---|---|
| Medium | 0 hrs | 0.5 hrs | 1 hr | 2 hrs | 4 hrs | 8 hrs | 12 hrs |
| 0.1N HCl | 0 | 22 | 33 | 49 | 70 | 97 | 102 |
| RSD %* | 0 | 3.2 | 2.7 | 1.8 | 1.0 | 0.6 | 0.6 |
| Range | 0 | 21–23 | 32–35 | 48–50 | 69–71 | 96–97 | 101–102 |
| 4% Ethanol | 0 | 22 | 33 | 49 | 69 | 96 | 102 |
| RSD %* | 0 | 3.3 | 3.0 | 2.5 | 2.0 | 1.6 | 1.8 |
| Range | 0 | 21–23 | 31–34 | 46–50 | 66–70 | 93–99 | 99–106 |
| 20% | 0 | 18 | 28 | 42 | 61 | 89 | 100 |
| Ethanol | 0 | 2.1 | 2.4 | 2.5 | 2.9 | 2.0 | 1.9 |
| % RSD* | 0 | 17–18 | 27–29 | 40–45 | 59–66 | 86–93 | 97–103 |
| 40% | 0 | 15 | 24 | 37 | 54 | 78 | 94 |
| Ethanol | 0 | 6.0 | 2.2 | 1.8 | 1.9 | 2.3 | 3.2 |
| RSD %* | 0 | 14–18 | 23–25 | 35–38 | 52–56 | 74–81 | 90–101 |

*RSD = Relative Standard Deviation

The presence of up to 40% ethanol did not significantly affect the dissolution profile of sustained release 40 mg oxymorphone tablets. The presence of 4% ethanol had an insignificant effect on the dissolution profile of 40 mg sustained release oxymorphone tablets compared to their dissolution profile in the absence of ethanol. Oxymorphone release was inversely correlated with the amount of ethanol in the dissolution medium. Presence of 20% and 40% ethanol in the dissolution medium slowed down the release of oxymorphone, which was still released in a controlled manner. No dose dumping was observed at concentrations of ethanol between 0% and 40%. Therefore, tablets with sustained release formulations described herein release oxymorphone in a controlled manner in the presence of up to at least 40% ethanol.

TABLE 10B

| | Similarity factor (f₂) for dissolution | | |
|---|---|---|---|
| | profiles of 40 mg oxymorphone | | |
| | sustained release tablets in | | |
| | 0.1N HCl and ethanol solutions | | |
| Medium | 4% ethanol | 20% ethanol | 40% ethanol |
| Relative to | 97 | 60 | 45 |
| 0.1N HCl | | | |

Similarity factors for ethanol-containing media relative to 0.1N HCl medium (0% ethanol) were 97, 60 and 45 for the 4%, 20% and 40% ethanol solutions, respectively. Thus, oxymorphone tablets resist beverage strength concentrations of ethanol and do not dose dump in the presence of at least up to 40% ethanol.

### Example 9

### Effect of Ethanol on Bioavailability of Oxymorphone from Sustained Release Oxymorphone Tablets

Healthy volunteers were used in a study to assess the pharmacokinetics of oxymorphone 40 mg sustained release tablets when co-administered with 240 mL of 40%, 20%, 4%, and 0% (water) ethanol.

The study design was a randomized, open-label, single-dose, four-period crossover in 28 subjects. To block the opioid effects of oxymorphone, naltrexone HCl (50 mg) was administered approximately 12 and 2 hours prior to each oxymorphone administration, and again at 12 hours after administration. Subjects were fasted overnight for at least 8 hours prior to dosing. Water was allowed ad lib except from 1 hour before dosing until 1 hour after dosing. A standardized meal was served 4 hours and 10 hours after dosing.

Oxymorphone 40 mg sustained release tablets were administered on four separate occasions with 240 mL of: A) 40% ethanol, B) 20% ethanol, C) 4% ethanol, or D) 0% ethanol. Serial blood samples were obtained from 0 to 48 hours after dosing. Plasma samples were assayed for oxymorphone. Pharmacokinetic parameters for oxymorphone were determined using non-compartmental methods for data evaluation. Point estimates and 90% confidence intervals (CIs) for natural logarithmic transformed Cₘₐₓ, AUC₀₋ₜ, and AUC_{0-inf} were calculated using Least Squares Means (LSMeans). Any treatment in which a subject vomited during the dosing interval (0-12 hours) was excluded from the primary pharmacokinetic analysis.

Thirty subjects were enrolled in the study. Twenty-five subjects completed the study, meaning these subjects received all four treatments. Subjects who vomited within the dosing interval (0-12 hours) were to have that treatment excluded from the pharmacokinetic analysis. There were 10 subjects who vomited between 0-12 hours on treatment A (40% ethanol) and 5 subjects who vomited between 0-12 hours on treatment B (20%) ethanol. There were no subjects who vomited on treatments C (4% ethanol) or D (0% ethanol). Mean plasma concentration-time data for each treatment, excluding subject data from a treatment if the subject vomited, are shown in Table 11.

TABLE 11
Mean oxymorphone plasma concentrations
(excluding subjects with emesis) [pg/ml]

| Time | | 0% ethanol | 4% ethanol | 20% ethanol | 40% |
|---|---|---|---|---|---|
| (hr) | | (N = 25) | (N = 25) | (N = 20) | ethanol (N = 15) |
| 0 | hr | 0.000 | 4.200 | 1.115 | 0.000 |
| 0.25 | hr | 316.248 | 269.400 | 255.910 | 686.880 |
| 0.5 | hr | 1218.988 | 1067.048 | 1307.611 | 1968.407 |
| 0.75 | hr | 1572.360 | 1469.992 | 2067.158 | 2520.593 |
| 1 | hr | 1716.480 | 1556.372 | 2135.500 | 2630.867 |
| 1.5 | hrs | 1726.720 | 1785.560 | 2352.500 | 2746.200 |
| 2 | hrs | 1930.840 | 1944.920 | 2442.000 | 2466.000 |
| 3 | hrs | 1694.800 | 1854.040 | 2179.750 | 2556.667 |
| 4 | hrs | 1450.800 | 1754.880 | 1838.400 | 2416.000 |
| 5 | hrs | 1800.600 | 2002.400 | 1768.700 | 2402.533 |
| 6 | hrs | 1681.080 | 1877.440 | 1591.350 | 1944.933 |
| 8 | hrs | 1262.880 | 1517.480 | 1359.550 | 1061.200 |
| 10 | hrs | 1002.800 | 1187.000 | 1162.000 | 889.200 |
| 12 | hrs | 1429.316 | 1489.280 | 1420.050 | 1223.667 |
| 16 | hrs | 876.800 | 872.760 | 958.400 | 854.067 |
| 24 | hrs | 443.872 | 451.920 | 403.305 | 407.933 |
| 36 | hrs | 254.988 | 238.020 | 241.980 | 261.647 |
| 48 | hrs | 95.180 | 99.976 | 85.675 | 116.207 |

Statistical analyses of the pharmacokinetic parameters are presented in Table 12.

TABLE 12

| | Oxymorphone treatment | | | |
|---|---|---|---|---|
| | (excluding subjects that vomited) | | | |
| Pharmacokinetic | 40% | | | 0% |
| parameter (SD) | ethanol | 20% ethanol | 4% ethanol | ethanol |
| Cₘₐₓ, pg/mL | 3917 | 3089 | 2564 | 2373 |
| | (1672) | (1150) | (1037) | (870) |
| Tₘₐₓ, h | 1.50 | 1.50 | 3.0 | 2.0 |
| | (0.75–6.0) | (0.75–8.0) | (1.0–12.0) | (0.5–12.0) |
| AUC_{0–t}, pg · h/mL | 36385 | 35389 | 35146 | 33350 |
| | (12441) | (11495) | (12534) | (11864) |
| AUC_{0–inf}, pg · h/mL | 39973^{α} | 36889 | 37551^{b} | 36034^{b} |
| | (13595) | (12356) | (13452) | (11388) |
| t_{1/2}, h | 11.3^{α} | 9.9 | 10.4^{b} | 10.7^{b} |
| | (3.5) | (3.2) | (4.1) | (4.7) |
| N | 15 | 20 | 25 | 25 |

Median and range reported for Tₘₐₓ
^{α}n = 13
^{b}n = 24

Geometric mean ratios (GMR) and 90% CI for those treatments in which subjects completed the study without vomiting between 0-12 hours are shown in Table 13.

TABLE 13

| | Oxymorphone treatment | | | | | |
|---|---|---|---|---|---|---|
| Pharma- | 40% ethanol/ | | 20% ethanol/ | | 4% ethanol/ | |
| cokinetic | 0% ethanol | | 0% ethanol | | 0% ethanol | |
| Parameter | Ratio | 90% CI | Ratio | 90% CI | Ratio | 90% CI |
| Cₘₐₓ | 1.703 | 1.476, 1.966 | 1.309 | 1.151, 1.488 | 1.073 | 0.952, |
| | | | | | | 1.209 |
| AUC_{0–t} | 1.129 | 1.03, 1.24 | 1.040 | 0.95, 1.13 | 1.055 | 0.97, 1.14 |
| AUC_{0–inf} | 1.127 | 1.03, 1.24 | 1.010 | 0.93, 1.09 | 1.022 | 0.95, 1.10 |

The mean plasma concentration-time data in Table 11 show that the 40% and 20% ethanol treatments produce higher plasma concentrations during the first 4 to 6 hours compared to the 0% ethanol treatment. The 4% ethanol treatment mean plasma concentrations were similar to those for the 0% ethanol treatment. All data were comparable from 16 to 48 hours after dosing. Secondary peaks were observed at 5 hours for the 4% and 0% ethanol treatments and 12 hours for all four treatments. Although the 40% ethanol treatment mean plasma concentration was higher than 0%, 4%, or 20% from 0.5 to 6 hours, the concentration then declined and was lower than the other three treatments at 8 to 12 hours. Cₘₐₓ was the only pharmacokinetic parameter that appeared to be directly related to the ethanol treatment (Table 12). From the ratios shown in Table 13, it can be seen that the increases in Cₘₐₓ were 70%, 31%, and 7% for the 40% ethanol, 20% ethanol and 4% ethanol treatments, respectively, compared to the 0% ethanol treatment. Changes in AUC₀₋ₜ and AUC_{0-inf} ranged from 1% to 13% for the ethanol treatments compared to 0% ethanol (Table 13). Other than Cₘₐₓ, no significant differences for the pharmacokinetic parameters were observed among various treatments.

Analysis of all subjects regardless of whether they vomited is presented in Tables 14 and 15. Mean plasma concentration-time data for each treatment, without any exclusions for vomiting, are shown in Table 14.

TABLE 14

| | | Mean oxymorphone plasma concentrations | | | |
|---|---|---|---|---|---|
| | | (including subjects who vomited) [pg/ml] | | | |
| Time | | 0% ethanol | 4% ethanol | 20% ethanol | 40% |
| (hr) | | (N = 25) | (N = 25) | (N = 25) | ethanol (N = 25) |
| 0 | hr | 0.000 | 4.200 | 0.892 | 0.000 |
| 0.25 | hr | 316.248 | 269.400 | 205.892 | 544.828 |
| 0.5 | hr | 1218.988 | 1067.048 | 1090.458 | 1775.428 |
| 0.75 | hr | 1572.360 | 1469.992 | 1718.917 | 2641.636 |
| 1 | hr | 1716.480 | 1556.372 | 1860.552 | 2640.640 |
| 1.5 | hrs | 1726.720 | 1785.560 | 2045.680 | 2481.396 |
| 2 | hrs | 1930.840 | 1944.920 | 2138.240 | 2208.060 |
| 3 | hrs | 1694.800 | 1854.040 | 1981.320 | 2166.160 |
| 4 | hrs | 1450.800 | 1754.880 | 1720.920 | 2152.960 |
| 5 | hrs | 1800.600 | 2002.400 | 1695.680 | 2635.628 |
| 6 | hrs | 1681.080 | 1877.440 | 1481.040 | 2311.740 |
| 8 | hrs | 1262.880 | 1517.480 | 1226.040 | 1259.644 |
| 10 | hrs | 1002.800 | 1187.000 | 1024.568 | 866.844 |
| 12 | hrs | 1429.316 | 1489.280 | 1250.080 | 981.016 |
| 16 | hrs | 876.800 | 872.760 | 844.264 | 692.216 |
| 24 | hrs | 443.872 | 451.920 | 359.224 | 338.700 |
| | | 254.988 | 238.020 | 227.056 | 233.728 |
| | | 95.180 | 99.976 | 80.784 | 97.752 |

Mean plasma concentration-time profiles without excluding treatments (n=25) in which subjects vomited (Table 14), showed the 40% ethanol treatment with a secondary peak at 5 hours, which was not clearly evident in Table 11, where only 15 subjects were represented. The 20% ethanol treatment (n=25) appeared to be similar to that of Table 11, where there were 20 subjects. The 4% and 0% ethanol treatments represented the same sample of subjects as those in Table 11. As previously indicated in Table 12, Cₘₐₓ was the only pharmacokinetic parameter that appeared to be directly related to the ethanol treatment (Table 15).

TABLE 15

| | Oxymorphone treatment | | | |
|---|---|---|---|---|
| Mean | (including subjects who vomited, N = 25) | | | |
| Pharmacokinetic | 40% | | | |
| Parameter (SD) | ethanol | 20% ethanol | 4% ethanol | 0% ethanol |
| Cₘₐₓ, pg/mL | 4124 | 2815 | 2564 | 2373 |
| | (2251) | (1227) | (1037) | (870) |
| Tₘₐₓ, h | 1.50 | 2.0 | 3.0 | 2.0 |
| | (0.75–6.0) | (0.75–8.0) | (1.0–12.0) | (0.5–12.0) |
| AUC_{0–t}, pg h/ml | 33677 | 31815 | 35146 | 33350 |
| | (13772) | (13456) | (12533) | (11864) |
| AUC_{0–inf}, pg h/ml | 37128^{a} | 34677^{b} | 37551 | 36034 |
| | (14803) | (13432) | (13452) | (11388) |
| t_{1/2}, h | 11.7^{a} | 9.9^{b} | 10.4 | 10.7 |
| | (4.5) | (3.1) | (4.1) | (4.7) |
| N | 25 | 25 | 25 | 25 |

^{a}n = 22
^{b}n = 23

GMR data shown in Table 16 indicate that increases in Cₘₐₓ were 62%, 15%, and 8% for the 40% ethanol, 20% ethanol and 4% ethanol treatments, respectively, as compared to the 0% ethanol treatment. Changes in AUC₀₋ₜ, and AUC_{0-inf} ranged from −10% to 7% for the ethanol treatments as compared to 0% ethanol (Table 16). The 40% and 20% Cₘₐₓ, AUC₀₋₁, and AUC_{0-inf} increases were lower when subjects who vomited were included.

TABLE 16
Oxymorphone treatment (including subjects who vomited, N = 25)

| | 40% ethanol/ | | 20 ethanol/ | | 4% ethanol/ | |
|---|---|---|---|---|---|---|
| | 0% ethanol | | 0% ethanol | | 0% ethanol | |
| Parameter | Ratio | 90% CI | Ratio | 90% CI | Ratio | 90% CI |
| Cₘₐₓ | 1.623 | 1.365, 1.931 | 1.145 | 0.963, 1.362 | 1.077 | 0.905, |
| | | | | | | 1.281 |
| AUC_{0–t} | 0.961 | 0.79, 1.18 | 0.897 | 0.73, 1.10 | 1.070 | 0.87, 1.31 |
| AUC_{0–inf} | 0.953 | 0.78, 1.16 | 0.920 | 0.75, 1.12 | 1.034 | 0.85, 1.26 |

### Example 10

### Effect of Food on Bioavailability of 40 Mg Sustained Release Oxymorphone Tablets and 4×10 mg Oxymorphone Immediate Release Tablets

A study was performed in healthy volunteers to assess the effect of food on the bioavailability of sustained release 40 mg oxymorphone tablets and oxymorphone immediate release tablets (4×10 mg). The study design was a randomized, open-label, single-dose, four-period crossover in 28 subjects. The 40 mg oxymorphone sustained release tablet and 4×10 mg oxymorphone immediate release tablets were evaluated under fed and fasted conditions. To block the opioid effects of oxymorphone, naltrexone HCl (50 mg) was administered approximately 12 hours prior to each oxymorphone administration. Subjects were fasted overnight for at least 8 hours prior to dosing. For the fed treatment subjects were served a high-fat breakfast and were dosed 10 minutes after completion of the breakfast. Each dose was administered with 240 mL of water. Subjects were not permitted any other food until 4 hours after dosing. Serial blood samples were obtained from 0 to 72 hours after dosing. Plasma samples were assayed for oxymorphone. Pharmacokinetic parameters for oxymorphone were determined using non-compartmental methods. Point estimates and 90% CIs for natural logarithmic transformed Cₘₐₓ, AUC₀₋ₜ, and AUC_{0-inf} were calculated using LSMeans.

Twenty-five subjects completed the study. The mean plasma concentration-time data for the fasted and fed treatments for the sustained release tablet are shown in Table 17.

TABLE 17

| | |
|---|---|
| | Mean oxymorphone |
| | plasma concentrations |
| | 40 mg sustained release |
| Time | oxymorphone tablets [ng/ml] |

| (hr) | | Fasted | Fed |
|---|---|---|---|
| 0 | | 0.00 | 0.00 |
| 0.25 | hr | 0.47 | 0.22 |
| 0.50 | hr | 1.68 | 0.97 |
| 0.75 | hr | 1.92 | 1.90 |
| 1 | hr | 2.09 | 2.61 |
| 1.5 | hrs | 2.18 | 3.48 |
| 2 | hrs | 2.18 | 3.65 |
| 3 | hrs | 2.00 | 2.86 |
| 4 | hrs | 1.78 | 2.45 |
| 5 | hrs | 1.86 | 2.37 |
| 6 | hrs | 1.67 | 2.02 |
| 8 | hrs | 1.25 | 1.46 |
| 10 | hrs | 1.11 | 1.17 |
| 12 | hrs | 1.34 | 1.21 |
| 24 | hrs | 0.55 | 0.47 |
| 36 | hrs | 0.21 | 0.20 |
| 48 | hrs | 0.06 | 0.05 |
| 60 | hrs | 0.03 | 0.01 |
| 72 | hrs | 0.00 | 0.00 |

As shown in Table 17 the fed treatment produced higher plasma oxymorphone concentrations during the first 8 hours compared to the fasted treatment. The mean plasma concentrations for both treatments were similar from 10 to 48 hours after dosing. Secondary peaks were observed at 5 hours for the fasted treatment and at 12 hours both treatments. The mean plasma oxymorphone concentration-time data or the fasted and fed treatments for the immediate release tablets are shown in Table 18. The fed treatment produced higher plasma concentrations during the first 10 hours compared to the fasted treatment. The mean plasma concentrations for both treatments were similar from 12 to 48 hours after dosing. Secondary peaks were seen at 12 hours for the fasted and fed treatments.

Mean plasma oxymorphone concentration time profiles for the fed and fasted treatments for the immediate release oxymorphone tablets (4×10 mg) are shown in Table 18.

TABLE 18

Mean oxymorphone
plasma concentration
4 × 10 mg IR
oxymorphone tablets
[ng/ml]

| Time (hr) | | Fasted | Fed |
|---|---|---|---|
| 0 | | 0.00 | 0.00 |
| 0.25 | hr | 3.34 | 1.79 |
| 0.50 | hr | 7.28 | 6.59 |
| 0.75 | hr | 6.60 | 9.49 |
| 1 | hr | 6.03 | 9.91 |
| 1.5 | hrs | 4.67 | 8.76 |
| 2 | hrs | 3.68 | 7.29 |
| 3 | hrs | 2.34 | 4.93 |
| 4 | hrs | 1.65 | 3.11 |
| 5 | hrs | 1.48 | 2.19 |
| 6 | hrs | 1.28 | 1.71 |
| 8 | hrs | 0.92 | 1.28 |
| 10 | hrs | 0.78 | 1.09 |
| 12 | hrs | 1.04 | 1.24 |
| 24 | hrs | 0.40 | 0.44 |
| 36 | hrs | 0.16 | 0.18 |
| 48 | hrs | 0.04 | 0.05 |
| 60 | hrs | 0.01 | 0.01 |
| 72 | hrs | 0.00 | 0.00 |

The fed treatment with 4×10 mg immediate release oxymorphone tablets produced higher plasma oxymorphone concentrations during the first 10 hours compared to the fasted treatment. The mean plasma oxymorphone concentrations for both treatments were similar from 12 to 48 hours after dosing. Secondary peaks were observed at 12 hours for the fasted treatment and fed treatments. Cₘₐₓ was increased in the presence of food for both the sustained release and the immediate release tablets and AUC was increased by food for the immediate release tablets (Table 19). From the GMR data (Table 20) it can be seen that food increased Cₘₐₓ by 51% and 38% for the sustained release and immediate release tablets, respectively, when compared to administration under fasted conditions. Food increased AUC₀₋ₜ and AUC_{0-inf} by 43% and 38%, respectively for the immediate release tablets. For the sustained release tablet administered with food, the AUC₀₋ₜ and AUC_{0-inf} increases were less than 10% and the 90% CIs were within 80-125%.

TABLE 19
Oxymorphone treatment (N = 25)

| Mean | 40 mg sustained | | 4 × 10 mg immediate | |
|---|---|---|---|---|
| Pharmacokinetic | release tablet | | release tablets | |
| Parameter (SD) | Fed | Fasted | Fed | Fasted |
| Cₘₐₓ, pg/mL | 4250 | 2790 | 12090 | 9070 |
| | (1210) | (840) | (5420) | (4090) |
| Tₘₐₓ, h | 2.00 | 1.00 | 1.00 | 0.50 |
| | (0.5–5.0) | (0.5–12.0) | (0.25–3.0) | (0.25–2.0) |
| AUC_{0–t}, pg · h/mL | 38200 | 35700 | 51350 | 36000 |
| | (11040) | (10580) | (20200) | (12520) |
| AUC_{0–inf}, pg · h/mL | 41170 | 40620 | 54100 | 39040 |
| | (10460) | (11380) | (20260) | (12440) |
| t_{1/2}, h | 10.5 | 12.2 | 9.6 | 11.7 |
| | (5.5) | (7.6) | (3.6) | (6.2) |

Median and range reported for Tₘₐₓ

TABLE 20
Oxymorphone treatment

| | 40 mg sustained | | 4 × 10 mg immediate | |
|---|---|---|---|---|
| Pharma- | release tablet | | release tablet | |
| cokinetic | Ratio | | Ratio | |
| parameter | (fed/fasted) | 90% CI | (fed/fasted) | 90% CI |
| Cₘₐₓ | 1.507 | 1.3777, 1.6970 | 1.376 | 1.156, 1.637 |
| AUC_{0–t} | 1.07 | 0.94, 1.22 | 1.43 | 1.32, 1.55 |
| AUC_{0–inf} | 1.02 | 0.91, 1.15 | 1.38 | 1.28, 1.41 |

From the GMR data (Table 20) it can be seen that food increased Cₘₐₓ by 51% and 38% for the sustained release and immediate release tablets, respectively, when compared to administration under fasted conditions. Food increased AUC₀₋ₜ and AUC_{0-inf} by 43% and 38%, respectively for the immediate release tablets. For the sustained release tablet, the AUC₀₋ₜ and AUC_{-inf} increases with food were small and the 90% CIs were within 80-125%.

The in vitro study (Example 8) showed that 40% ethanol did not increase the dissolution rate of the oxymorphone sustained release 40 mg tablet. These data indicate that the formulation drug release matrix is not compromised by beverage-strength ethanol concentrations and the premature release of oxymorphone in vivo when exposed to ethanol at concentrations up to 40% does not occur. However, the data from the human ethanol study demonstrated that co-administration of 240 mL of 40% ethanol, and to a lesser extent 20% ethanol, increased the Cₘₐₓ of oxymorphone from the 40 mg sustained release tablet while having no demonstrable effect on the AUC (Tables 12 and 13). The in vitro and in vivo results suggest that beverage-strength ethanol does not directly effect the integrity of formulation, but may cause other effect(s), that can lead to an apparent increased rate of absorption of oxymorphone.

Interestingly, an increased rate of absorption of oxymorphone is also observed when oxymorphone 40 mg sustained release tablets are administered after a high-fat meal (Tables 19 and 20). The magnitude of the increase and the plasma concentration-time course are similar when oxymorphone tablets formulated with TIMERx-N® are administered after a high-fat meal or with ethanol (see Tables 11 and 16). This observation suggests that there may be a common mechanism between food and ethanol leading to the increase in Cₘₐₓ. The pharmacokinetic parameters measured following dosing of oxymorphone immediate release tablets and oral solutions were also affected when taken after a high-fat meal (Tables 19 and 20). In addition to an increase in Cₘₐₓ, the AUC for the immediate release tablets also increased, unlike the results for the sustained release tablets, where AUC did not change appreciably after ethanol or food. These differences suggest that the sustained release tablets are not releasing oxymorphone at an accelerated rate in the presence of ethanol, but that it is only the level of oxymorphone dissolved in the gastrointestinal tract that is affected by the food or ethanol.

The in vitro results indicate no oxymorphone sustained release formulation-ethanol interaction. The results from the bioavailability study demonstrated that there is a pharmacokinetic interaction when 40 mg oxymorphone sustained release tablet is consumed with 240 mL of 40% ethanol, which represents an excessive intake of ethanol, with resultant increases in peak plasma concentrations similar to those observed when oxymorphone sustained release tablets are taken after a standardized high-fat meal. The underlying mechanism of this phenomenon is not clear at present.

Based on evaluation of the in vitro and earlier in vivo data, the increases in Cₘₐₓ observed are not believed to be caused by early release of oxymorphone owing to disintegration of the sustained release delivery system (i.e., dose dumping), but instead by an apparent increased rate of absorption, which is independent of the formulation.

Similar results are expected to be obtained with other drugs, because the properties of the sustained release system affect the dissolution properties of the formulation to a significantly larger extent than the nature of the drug in the formulation. Ethanol dissolution testing is contemplated to become a standard procedure in the development of new sustained release products.

The patents, patent applications, and publications cited herein are incorporated by reference herein in their entirety.

Various modifications of the invention, in addition to those described herein, will be apparent to one skilled in the art from the foregoing description. Such modifications are intended to fall within the scope of the appended claims.

What is claimed is:

1. A sustained release formulation comprising:
a drug; and
a sustained release delivery system comprising a hydrophilic gum, a homopolysaccharide gum, and a pharmaceutical diluent,
wherein at least about 30% of the hydrophilic gum used to make the sustained release formulation can pass through a #270 mesh sieve, and the sustained release formulation releases less than about 70% of the drug within 2 hours after ingestion with either an ethanol-free or an ethanol-containing beverage.

2. The sustained release formulation of claim 1, wherein the hydrophilic compound is xanthan gum.

3. The sustained release formulation of claim 1, wherein the sustained release delivery system further comprises a hydrophobic polymer.

4. The sustained release formulation of claim 1, wherein the sustained release delivery system further comprises a cationic cross-linking compound selected from monovalent cations, multivalent cations, and salts.

5. The sustained release formulation of claim 1, further comprising an outer coating, wherein the outer coating comprises a hydrophobic polymer.

6. The sustained release formulation of claim 1, further comprising an outer coating, wherein the outer coating comprises a plasticizer.

7. The sustained release formulation of claim 1 or 2, wherein the drug is selected from the group consisting of alprazolam, lithium carbonate, divalproex sodium, neutral sulfate salts of dextroamphetamine and amphetamine with the dextro isomer of amphetamine saccharate and d,l-amphetamine aspartate monohydrate, tramadol hydrochloride, and other pharmaceutically acceptable salt of the active pharmaceutical ingredient thereof.

8. The sustained release formulation of claim 1 or 2, wherein the drug is an opioid.

9. The sustained release formulation of claim 8, wherein the opioid is a mu-agonist or a mixed mu-agonist/antagonist.

10. The sustained release formulation of claim 9, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papaveretum, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, an ether thereof, an ester thereof, and a derivative thereof.

11. The sustained release formulation of claim 10, wherein the opioid is oxymorphone.

12. A sustained release formulation comprising:
a drug; and
a sustained release delivery system comprising a hydrophilic gum, a cationic cross-linking compound selected from monovalent cations, multivalent cations and salts, and a pharmaceutical diluent,
wherein at least about 30% of the hydrophilic gum used to make the sustained release formulation can pass through a #270 mesh sieve, and the sustained release formulation releases less than about 70% of the drug within 2 hours after ingestion with either an ethanol-free or an ethanol-containing beverage.

13. The sustained release formulation of claim 12, wherein the hydrophilic compound is xanthan gum.

14. The sustained release formulation of claim 12, wherein the sustained release delivery system further comprises a hydrophobic polymer.

15. The sustained release formulation of claim 12, further comprising an outer coating, wherein the outer coating comprises a hydrophobic polymer.

16. The sustained release formulation of claim 12, further comprising an outer coating, wherein the outer coating comprises a plasticizer.

17. The sustained release formulation of claim 12 or 13, wherein the drug is selected from the group consisting of alprazolam, lithium carbonate, divalproex sodium, neutral sulfate salts of dextroamphetamine and amphetamine with the dextro isomer of amphetamine saccharate and d,l-amphetamine aspartate monohydrate, tramadol hydrochloride, and other pharmaceutically acceptable salt of the active pharmaceutical ingredient thereof.

18. The sustained release formulation of claim 12 or 13, wherein the drug is an opioid.

19. The sustained release formulation of claim 18, wherein the opioid is a mu-agonist or a mixed mu-agonist/antagonist.

20. The sustained release formulation of claim 19, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papaveretum, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, an ether thereof, an ester thereof, and a derivative thereof.

21. The sustained release formulation of claim 20, wherein the opioid is oxymorphone.

22. A sustained release formulation comprising:
a drug; and
a sustained release delivery system comprising a hydrophilic gum, a homopolysaccharide gum, and a pharmaceutical diluent,
wherein at least about 30% of the hydrophilic gum particles used to make the sustained release formulation are smaller than about 53 microns in diameter, and the sustained release formulation releases less than 70% of the drug within 2 hours after ingestion with either an ethanol-free or an ethanol-containing beverage.

23. The sustained release formulation of claim 22, wherein the hydrophilic gum is xanthan gum.

24. The sustained release formulation of claim 22, wherein the sustained release delivery system further comprises a hydrophobic polymer.

25. The sustained release formulation of claim 22, wherein the sustained release delivery system further comprises a cationic cross-linking compound selected from monovalent cations, multivalent cations, and salts.

26. The sustained release formulation of claim 22, further comprising an outer coating, wherein the outer coating comprises a hydrophobic polymer.

27. The sustained release formulation of claim 22, further comprising an outer coating, wherein the outer coating comprises a plasticizer.

28. The sustained release formulation of claim 22 or 23, wherein the drug is selected from the group consisting of alprazolam, lithium carbonate, divalproex sodium, neutral sulfate salts of dextroamphetamine and amphetamine with the dextro isomer of amphetamine saccharate and d,l-amphetamine aspartate monohydrate, tramadol hydrochloride, and other pharmaceutically acceptable salts thereof.

29. The sustained release formulation of claim 22 or 23, wherein the drug is an opioid.

30. The sustained release formulation of claim 29, wherein the opioid is a mu-agonist or a mixed mu-agonist/antagonist.

31. The sustained release formulation of claim 30, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papaveretum, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, an ether thereof, an ester thereof, and a derivative thereof.

32. The sustained release formulation of claim 31, wherein the opioid is oxymorphone.

33. A sustained release formulation comprising:
a drug; and
a sustained release delivery system comprising a hydrophilic gum, a cationic cross-linking compound selected from monovalent cations, multivalent cations and salts, and a pharmaceutical diluent,
wherein at least about 30% of the hydrophilic gum particles used to make the sustained release formulation are smaller than about 53 microns in diameter, and the sustained release formulation releases less than 70% of the drug within 2 hours after ingestion with either an ethanol-free or an ethanol-containing beverage.

34. The sustained release formulation of claim 33, wherein the hydrophilic gum is xanthan gum.

35. The sustained release formulation of claim 33, wherein the sustained release delivery system further comprises a hydrophobic polymer.

36. The sustained release formulation of claim 33, further comprising an outer coating, wherein the outer coating comprises a hydrophobic polymer.

37. The sustained release formulation of claim 33, further comprising an outer coating, wherein the outer coating comprises a plasticizer.

38. The sustained release formulation of claim 33 or 34, wherein the drug is selected from the group consisting of alprazolam, lithium carbonate, divalproex sodium, neutral sulfate salts of dextroamphetamine and amphetamine with the dextro isomer of amphetamine saccharate and d,l-amphetamine aspartate monohydrate, tramadol hydrochloride, and other pharmaceutically acceptable salt of the active pharmaceutical ingredient thereof.

39. The sustained release formulation of claim 33 or 34, wherein the drug is an opioid.

40. The sustained release formulation of claim 39, wherein the opioid is a mu-agonist or a mixed mu-agonist/antagonist.

41. The sustained release formulation of claim 40, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papaveretum, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, an ether thereof, an ester thereof, and a derivative thereof.

42. The sustained release formulation of claim 41, wherein the opioid is oxymorphone.

43. A method for making a sustained release formulation comprising:
a drug; and
a sustained release delivery system,
wherein the sustained release delivery system comprises a hydrophilic gum, a homopolysaccharide gum, and a pharmaceutical diluent, the method comprising:
providing the hydrophilic gum with at least 30% of particles less than about 53 microns in diameter;
granulating the hydrophilic gum, the homopolysaccharide gum and the pharmaceutical diluent to form granules;
mixing the granules with the drug to form a granulated composition; and
applying pressure to the granulated composition to make the formulation.

44. The method of claim 43, wherein the hydrophilic gum is xanthan gum.

45. The method of claim 43, wherein providing comprises receiving the hydrophilic gum.

46. The method of claim 43, wherein providing comprises manufacturing the hydrophilic gum.

47. The method of claim 43, wherein providing comprises processing the hydrophilic gum.

48. The method of claim 47, wherein processing comprises measuring the size of at least a fraction of the polysaccharide gum particles.

49. The method of claim 47, wherein processing comprises passing at least a fraction of the hydrophilic gum through a sieve.

50. The method of claim 49, wherein the sieve is a #270 mesh sieve.

51. The method of claim 43, further comprising applying an outer coating onto at least part of the sustained release formulation.

52. The method of claim 51, wherein the outer coating comprises a hydrophobic polymer.

53. The method of claim 51, wherein the outer coating comprises a plasticizer.

54. The method of claim 43, wherein granulating comprises mixing ingredients with a solution comprising water.

55. The method of claim 43, wherein granulating comprises mixing ingredients with an alcohol solution.

56. The method of claim 55, wherein the alcohol solution comprises ethanol.

57. The method of claim 43, wherein the sustained release delivery system further comprises a cationic cross-linking compound selected from monovalent cations, multivalent cations, and salts.

58. The method of claim 43 or 44, wherein the drug is selected from the group consisting of alprazolam, lithium carbonate, divalproex sodium, neutral sulfate salts of dextroamphetamine and amphetamine with the dextro isomer of amphetamine saccharate and d,l-amphetamine aspartate monohydrate, tramadol hydrochloride, and other pharmaceutically acceptable salt of the active pharmaceutical ingredient thereof.

59. The method of claim 43 or 44, wherein the drug is an opioid.

60. The method of claim 59, wherein the opioid is a mu-agonist or a mixed mu-agonist/antagonist.

61. The method of claim 60, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papaveretum, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, an ether thereof, an ester thereof, and a derivative thereof.

62. The method of claim 43, further comprising recording a dissolution profile of the formulation in an ethanol-containing solution.

63. The method of claim 62, wherein the opioid is oxymorphone.

64. A method for making a sustained release formulation comprising:
a drug; and
a sustained release delivery system,
wherein the sustained release delivery system comprises a hydrophilic gum, a cationic cross-linking compound selected from monovalent cations, multivalent cations and salts, and a pharmaceutical diluent, the method comprising:
providing the hydrophilic gum with at least 30% of particles less than about 53 microns in diameter;
granulating the hydrophilic gum, the homopolysaccharide gum and the pharmaceutical diluent to form granules;
mixing the granules with the drug to form a granulated composition; and
applying pressure to the granulated composition to make the formulation.

65. The method of claim 64, wherein the hydrophilic gum is xanthan gum.

66. The method of claim 64, wherein providing comprises receiving the hydrophilic gum.

67. The method of claim 64, wherein providing comprises manufacturing the hydrophilic gum.

68. The method of claim 64, wherein providing comprises processing the least one hydrophilic gum.

69. The method of claim 68, wherein processing comprises measuring the size of at least a fraction of the hydrophilic gum particles.

70. The method of claim 68, wherein processing comprises passing at least a fraction of the hydrophilic gum through a sieve.

71. The method of claim 70, wherein the sieve is a #270 mesh sieve.

72. The method of claim 64, further comprising applying an outer coating onto at least part of the sustained release formulation.

73. The method of claim 72, wherein the outer coating comprises a hydrophobic polymer.

74. The method of claim 72, wherein the outer coating comprises a plasticizer.

75. The method of claim 64, wherein granulating comprises mixing ingredients with a solution comprising water.

76. The method of claim 64, wherein granulating comprises mixing ingredients with an alcohol solution.

77. The method of claim 76, wherein the alcohol solution comprises ethanol.

78. The method of claim 64 or 65, wherein the drug is selected from the group consisting of alprazolam, lithium carbonate, divalproex sodium, neutral sulfate salts of dextroamphetamine and amphetamine with the dextro isomer of amphetamine saccharate and d,l-amphetamine aspartate monohydrate, tramadol hydrochloride, and other pharmaceutically acceptable salt of the active pharmaceutical ingredient thereof.

79. The method of claim 64 or 65, wherein the drug is an opioid.

80. The method of claim 79, wherein the opioid is a mu-agonist or a mixed mu-agonist/antagonist.

81. The method of claim 80, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papaveretum, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, an ether thereof, an ester thereof, and a derivative thereof.

82. The method of claim 81, wherein the opioid is oxymorphone.

83. The method of claim 64, further comprising recording a dissolution profile of the sustained release formulation or a solid dosage form comprising the sustained release formulation in an ethanol-containing solution.

84. A method for making a sustained release formulation comprising:
a drug; and
a sustained release delivery system,
wherein the sustained release delivery system comprises a hydrophilic gum, a homopolysaccharide gum, and a pharmaceutical diluent, the method comprising:
mixing the hydrophilic gum of average and/or mean particle size larger than about 53 microns in diameter, the homopolysaccharide gum and the pharmaceutical diluent with a solution comprising water to form granules;
mixing the granules with drug to form a granulated composition; and
applying pressure to the granulated composition to make the formulation.

85. The method of claim 84, wherein the hydrophilic gum is xanthan gum.

86. The sustained release formulation of claim 84, wherein the sustained release delivery system further comprises a cationic cross-linking compound selected from monovalent cations, multivalent cations, and salts.

87. The method of claim 84, further comprising applying an outer coating onto at least part of the sustained release formulation.

88. The method of claim 84, wherein the drug is selected from the group consisting of alprazolam, lithium carbonate, divalproex sodium, neutral sulfate salts of dextroamphetamine and amphetamine with the dextro isomer of amphetamine saccharate and d,l-amphetamine aspartate monohydrate, tramadol hydrochloride, and other pharmaceutically acceptable salt of the active pharmaceutical ingredient thereof.

89. The method of claim 84 or 85, wherein the drug is an opioid.

90. The method of claim 89, wherein the opioid is a mu-agonist or a mixed mu-agonist/antagonist.

91. The method of claim 90, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papaveretum, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, an ether thereof, an ester thereof, and a derivative thereof.

92. The method of claim 91, wherein the opioid is oxymorphone.

93. The method of claim 84, further comprising recording a dissolution profile of the sustained release formulation or a solid dosage form comprising the sustained release formulation in an ethanol-containing solution.

94. A method for making a sustained release formulation comprising:
a drug; and
a sustained release delivery system,
wherein the sustained release delivery system comprises a hydrophilic gum, a cationic cross-linking compound selected from monovalent cations, multivalent cations and salts, and pharmaceutical diluent, the method comprising:
mixing the hydrophilic gum of average and/or mean particle size larger than about 53 microns in diameter, the cationic cross-linking compound and the pharmaceutical diluent with a solution comprising water to form granules;
mixing the granules with the drug to form a granulated composition; and
applying pressure to the granulated composition to make the formulation.

95. The method of claim 94, hydrophilic gum is xanthan gum.

96. The method of claim 94, further comprising applying an outer coating onto at least part of the sustained release formulation.

97. The method of claim 94 or 95, wherein the drug is selected from the group consisting of alprazolam, lithium carbonate, divalproex sodium, neutral sulfate salts of dextroamphetamine and amphetamine with the dextro isomer of amphetamine saccharate and d,l-amphetamine aspartate monohydrate, tramadol hydrochloride, and other pharmaceutically acceptable salt of the active pharmaceutical ingredient thereof.

98. The method of claim 94 or 95, wherein the drug is an opioid.

99. The method of claim 98 wherein the opioid is a mu-agonist or a mixed mu-agonist/antagonist.

100. The method of claim 99, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papaveretum, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, an ether thereof, an ester thereof, and a derivative thereof.

101. The method of claim 100, wherein the opioid is oxymorphone.

102. A method for treating a patient having a condition comprising administering to the patient a therapeutically effective amount of the sustained release formulation of any one of claims 1, 2, 12, 13, 22, 23, 33 and 34.

103. A method for relieving pain comprising administering to a patient a therapeutically effective amount of the sustained release formulation of any one of claims 10, 11, 20, 21, 31, 32, 41, and 42.

104. A method for reducing dose dumping of a sustained release drug formulation comprising providing a patient a sustained release formulation according to any one of claims 1, 12, 22 and 33.

105. A solid dosage form comprising the sustained release formulation according to any one of claims 10, 11, 20, 21, 31, 32, 41, and 42.

106. The solid dosage form of claim 105 comprising a powder.

107. The solid dosage form of claim 105 comprising a tablet.

108. The solid dosage form of claim 105 comprising a capsule.

109. A sustained release formulation comprising from about 5 to about 80 mg of oxymorphone hydrochloride and from about 80 mg to about 360 mg of a sustained release delivery system; wherein the sustained release delivery system comprises from about 8.3% to about 41.7% by weight locust bean gum, from about 8.3% to about 41.7% by weight xanthan gum wherein at least about 30% of the xanthan gum particles can pass through a #270 mesh sieve, from about 20% to about 55% by weight dextrose, from about 5% to about 20% by weight calcium sulfate dihydrate, and from about 2% to about 10% ethyl cellulose, and the sustained release formulation releases less than 70% of the drug within 2 hours after ingestion with either an ethanol-free or an ethanol-containing beverage.

110. The sustained release formulation of claim 109, comprising about 20 mg of oxymorphone hydrochloride.

111. The sustained release formulation of claim 109, comprising about 160 mg of a sustained release delivery system.

112. The sustained release formulation of claim 109, wherein the sustained release delivery system comprises about 25% locust bean gum, about 25% xanthan gum, about 35% dextrose, about 10% calcium sulfate dihydrate, and about 5% ethyl cellulose.

113. The sustained release formulation of claim 109, further comprising an outer coating.

114. A method for treating a patient suffering from pain comprising administering a therapeutically effective amount of the sustained release formulation of claim 109.

115. A sustained release formulation comprising from about 5 to about 80 mg of oxymorphone hydrochloride and from about 300 mg to about 420 mg of a sustained release delivery system; wherein the sustained release delivery system comprises from about 8.3% to about 41.7% by weight locust bean gum, from about 8.3% to about 41.7% by weight xanthan gum having at least about 30% of particles smaller than about 53 microns in diameter, from about 20% to about 55% by weight dextrose, from about 5% to about 20% by weight calcium sulfate dihydrate, and from about 2% to about 10% ethyl cellulose, and the sustained release formulation releases less than 70% of the drug within 2 hours after ingestion with either an ethanol-free or an ethanol-containing beverage.

116. The sustained release formulation of claim 115, comprising about 20 mg of oxymorphone hydrochloride.

117. The sustained release formulation of claim 115, comprising about 360 mg of a sustained release delivery system.

118. The sustained release formulation of claim 115, wherein the sustained release delivery system comprises about 25% locust bean gum, about 25% xanthan gum, about 35% dextrose, about 10% calcium sulfate dihydrate, and about 5% ethyl cellulose.

119. The sustained release formulation of claim 115, further comprising an outer coating.

120. A method for treating a patient suffering from pain comprising administering a therapeutically effective amount of the sustained release formulation of claim 115.

121. A solid dosage form comprising the sustained release formulation according to any one of claims 109-113 and 115-119.

122. The solid dosage form of claim 121 comprising a powder.

123. The solid dosage form of claim 121 comprising a tablet.

124. The solid dosage form of claim 121 comprising a capsule.

125. A method of preventing dose-dumping of a drug in the presence of ethanol comprising: providing a patient who could consume ethanol while being treated with the drug an effective amount of the drug in the form of an ethanol-resistant sustained release formulation comprising:
the drug; and
a sustained release delivery system,
the delivery system comprising at least one hydrophilic gum, at least one homopolysaccharide gum and at least one pharmaceutical diluent, wherein at least about 30% of the hydrophilic gum used to make the sustained release formulation can pass through a #270 mesh sieve, and the sustained release formulation releases less than about 70% of the drug within 2 hours after ingestion with either an ethanol-free or an ethanol-containing beverage.

126. The method of claim 125, wherein the patient has a history of substance abuse.

127. The method of claim 126, wherein the substance abuse is alcohol abuse.

128. The method of claim 126, wherein the substance abuse is drug abuse.

129. The method of claim 125, wherein the ethanol-resistant sustained release formulation is a solid dosage form.

130. The method of claim 125, wherein the hydrophilic gum is xanthan gum.

131. The method of claim 129, wherein the solid dosage form forms a gel matrix with muco-adhesive properties when crushed or powdered upon contact with a fluid.

132. The method of claim 129, wherein the solid dosage form forms a viscous solution when crushed or powdered upon contact with a fluid.

133. The method of claim 129, wherein the solid dosage form is a tablet.

134. The method of claim 125, wherein the sustained release delivery system further comprises at least one hydrophobic polymer in an amount of less than about 5% by weight.

135. The method of claim 125, wherein the delivery system further comprises at least one cationic cross-linking compound selected from monovalent cations, multivalent cations, and salts.

136. The method of claim 125, wherein the drug is an anti-depressant.

137. The method of claim 125, wherein the drug is a drug used to treat bipolar disorder, panic disorder, epilepsy, migraine, and/or attention deficit hyperactivity disorder.

138. The method of claim 125, wherein the drug is an opioid.

139. The method of claim 138, wherein the opioid is a mu-agonist or a mixed mu-agonist/antagonist.

140. The method of claim 138, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papaveretum, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, a salt thereof, an ether thereof, an ester thereof and a derivative thereof.

141. A method of preventing dose-dumping of a drug in the presence of beverage-strength ethanol comprising:
providing a patient who could consume ethanol while being treated with the drug an effective amount of the drug in the form of an ethanol-resistant sustained release formulation comprising:
the drug; and
a sustained release delivery system,
the delivery system comprising at least one hydrophilic gum, at least one cationic cross-linking compound selected from monovalent metal cations, multivalent metal cations and salts, and at least one pharmaceutical diluent, wherein at least about 30% of the hydrophilic gum used to make the sustained release formulation can pass through a #270 mesh sieve, and the sustained release formulation releases less than about 70% of the drug within 2 hours after ingestion with either an ethanol-free or an ethanol-containing beverage.

142. The method of claim 141, wherein the patient has a history of substance abuse.

143. The method of claim 142, wherein substance abuse is alcohol abuse.

144. The method of claim 142, wherein the substance abuse is drug abuse.

145. The method of claim 141, wherein the ethanol-resistant sustained release formulation is a solid dosage form.

146. The method of claim 141, wherein the hydrophilic gum is xanthan gum.

147. The method of claim 145, wherein the solid dosage form forms a gel matrix with muco-adhesive properties when crushed or powdered upon contact with a fluid.

148. The method of claim 145, wherein the solid dosage form forms a viscous solution when crushed or powdered upon contact with a fluid.

149. The method of claim 145, wherein the solid dosage form is a tablet.

150. The method of claim 141, wherein the sustained release delivery system further comprises at least one hydrophobic polymer in an amount of less than about 5% by weight.

151. The method of claim 141, wherein the at least one cationic cross-linking compound is a sodium salt.

152. The method of claim 141, wherein the drug is an anti-depressant.

153. The method of claim 141, wherein the drug is a drug used to treat bipolar disorder, panic disorder, epilepsy, migraine, and/or attention deficit hyperactivity disorder.

154. The method of claim 141, wherein the drug is an opioid.

155. The method of claim 154, wherein the opioid is a mu-agonist or a mixed mu-agonist/antagonist.

156. The method of claim 155, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papaveretum, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, a salt thereof, an ether thereof, an ester thereof and a derivative thereof.

157. A method of improving safety of a drug formulation comprising: providing a patient who could consume ethanol while being treated with the drug an effective amount of the drug in the form of an ethanol-resistant sustained release formulation comprising:
the drug; and
a sustained release delivery system,
the sustained release delivery system comprising at least one hydrophilic gum, at least one homopolysaccharide gum and at least one pharmaceutical diluent, wherein the improvement in safety is a result of controlled hydrophilic gum particle size and ethanol-resistant sustained release properties of the formulation.

158. The method of claim 157, wherein the patient has a history of substance abuse.

159. The method of claim 158, wherein the substance abuse is alcohol abuse.

160. The method of claim 158, wherein the substance abuse is drug abuse.

161. The method of claim 157, wherein the ethanol-resistant sustained release formulation is a solid dosage form.

162. The method of claim 157, wherein the hydrophilic gum is xanthan gum.

163. The method of claim 161, wherein the solid dosage form forms a gel matrix with muco-adhesive properties when crushed or powdered upon contact with a fluid.

164. The method of claim 161, wherein the solid dosage form forms a viscous solution when crushed or powdered upon contact with a fluid.

165. The method of claim 160, wherein the solid dosage form is a tablet.

166. The method of claim 157, wherein the sustained release delivery system further comprises at least one hydrophobic polymer in an amount of less than about 5% by weight.

167. The method of claim 157, wherein the delivery system further comprises at least one cationic cross-linking compound selected from monovalent cations, multivalent cations, and salts.

168. The method of claim 157, wherein the drug is an anti-depressant.

169. The method of claim 157, wherein the drug is a drug used to treat bipolar disorder, panic disorder, epilepsy, migraine, and/or attention deficit hyperactivity disorder.

170. The method of claim 157, wherein the drug is an opioid.

171. The method of claim 170, wherein the opioid is a mu-agonist or a mixed mu-agonist/antagonist.

172. The method of claim 171, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papaveretum, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, a salt thereof, an ether thereof, an ester thereof and a derivative thereof.

173. A method of improving safety of a drug formulation comprising: providing a patient who could consume ethanol while being treated with the drug an effective amount of the drug in the form of an ethanol-resistant sustained release formulation comprising:
the drug; and
a sustained release delivery system,
the delivery system comprising at least one hydrophilic gum, at least one cationic cross-linking compound selected from monovalent metal cations, multivalent metal cations and salts, and at least one pharmaceutical diluent, wherein the improvement in safety is a result of controlled hydrophilic gum particle size and ethanol-resistant sustained release properties of the formulation.

174. The method of claim 173, wherein the patient has a history of substance abuse.

175. The method of claim 174, wherein substance abuse is alcohol abuse.

176. The method of claim 174, wherein the substance abuse is drug abuse.

177. The method of claim 173, wherein the ethanol-resistant sustained release formulation is a solid dosage form.

178. The method of claim 173, wherein the hydrophilic gum is xanthan gum.

179. The method of claim 177, wherein the solid dosage form forms a gel matrix with muco-adhesive properties when crushed or powdered upon contact with a fluid.

180. The method of claim 177, wherein the solid dosage form forms a viscous solution when crushed or powdered upon contact with a fluid.

181. The method of claim 177, wherein the solid dosage form is a tablet.

182. The method of claim 173, wherein the sustained release delivery system further comprises at least one hydrophobic polymer in an amount of less than about 5% by weight.

183. The method of claim 173, wherein the at least one cationic cross-linking compound is a sodium salt.

184. The method of claim 173, wherein the drug is an anti-depressant.

185. The method of claim 173, wherein the drug is a drug used to treat bipolar disorder, panic disorder, epilepsy, migraine, and/or attention deficit hyperactivity disorder.

186. The method of claim 173, wherein the drug is an opioid.

187. The method of claim 186, wherein the opioid is a mu-agonist or a mixed mu-agonist/antagonist.

188. The method of claim 187, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normophine, norpipanone, opium, oxycodone, oxymorphone, 6-hydroxyoxymorphone, papaveretum, pentazocine, phenadoxone, phenomorphan, phenazocine, phenoperidine, piminodine, piritramide, propheptazine, promedol, properidine, propiram, propoxyphene, sufentanil, tramadol, tilidine, a stereoisomer thereof, a metabolite thereof, a salt thereof, an ether thereof, an ester thereof and a derivative thereof.
